(19) United States
(12) Patent Application Publication
(10) Pub. No.: US 2026/0262433 A1
(43) Pub. Date: Sep. 3, 2026

(54) METHOD OF PRODUCING PHOTOELECTRIC CONVERSION ELEMENT

(71) Applicant: CANON KABUSHIKI KAISHA, Tokyo (JP)

(72) Inventors: KOHEI MAKISUMI, Shizuoka (JP); KENTARO TANAKA, Shizuoka (JP); MICHITAKA KITAHARA, Shizuoka (JP); NOBUHIRO NAKAMURA, Shizuoka (JP); TSUTOMU NISHIDA, Shizuoka (JP)

(21) Appl. No.: 19/651,949

(22) Filed: Apr. 20, 2026

Related U.S. Application Data
(63) Continuation of application No. PCT/JP2024/038114, filed on Oct. 25, 2024.

(30) Foreign Application Priority Data
Oct. 27, 2023 (JP) 2023-184750
Oct. 27, 2023 (JP) 2023-184756
Oct. 27, 2023 (JP) 2023-184761
Dec. 21, 2023 (JP) 2023-216294
Dec. 21, 2023 (JP) 2023-216296
Dec. 21, 2023 (JP) 2023-216299
Feb. 16, 2024 (JP) 2024-022244
Feb. 16, 2024 (JP) 2024-022246
Feb. 16, 2024 (JP) 2024-022251
Aug. 27, 2024 (JP) 2024-145656
Oct. 23, 2024 (JP) 2024-186710

(51) Int. Cl. H10K 71/12; B60L 8/00; H02S 20/23; H10K 30/85; H10K 85/10; H10K 85/30; H10K 85/50; H10K 85/60

(52) U.S. Cl. CPC H10K 71/12; H10K 30/85; H10K 85/141; H10K 85/311; H10K 85/326; H10K 85/371; H10K 85/50; H10K 85/654; H10K 85/6572; B60L 8/003; H02S 20/23

(57) ABSTRACT

To provide a photoelectric conversion element that achieves both mass productivity and the uniformity of photoelectric conversion efficiency, provided is a method of producing a photoelectric conversion element, the method including: a step (A) of forming a photoelectric conversion layer containing a crystal having a perovskite structure by using a large-area film formation method; and a step (B) of forming a charge-transporting layer by applying a paint for a charge-transporting layer onto the surface of the photoelectric conversion layer and then drying the paint, wherein the paint for a charge-transporting layer contains a charge-transporting particle and an insulating resin.

### CROSS-REFERENCE TO RELATED APPLICATIONS

This application is a Continuation of International Patent Application No. PCT/JP2024/038114, filed Oct. 25, 2024, which claims the benefit of Japanese Patent Application No. 2023-184761, filed Oct. 27, 2023, Japanese Patent Application No. 2023-184756, filed Oct. 27, 2023, Japanese Patent Application No. 2023-184750, filed Oct. 27, 2023, Japanese Patent Application No. 2023-216294, filed Dec. 21, 2023, Japanese Patent Application No. 2023-216296, filed Dec. 21, 2023, Japanese Patent Application No. 2023-216299, filed Dec. 21, 2023, Japanese Patent Application No. 2024-022244, filed Feb. 16, 2024, Japanese Patent Application No. 2024-022251, filed Feb. 16, 2024, Japanese Patent Application No. 2024-022246, filed Feb. 16, 2024, Japanese Patent Application No. 2024-145656, filed Aug. 27, 2024, and Japanese Patent Application No. 2024-186710, filed Oct. 23, 2024, all of which are hereby incorporated by reference herein in their entirety.

### BACKGROUND

### Field of the Technology

The present disclosure relates to a method of producing a photoelectric conversion element.

### Description of the Related Art

In order to solve a problem of the depletion of fossil energy and a global environmental problem caused by the use of the fossil energy, investigations on a renewable and clean alternative energy source, such as solar energy, wind power, or water power, have been actively performed. In particular, an interest in a solar cell that directly changes sunlight into electrical energy has been increasing. The term “solar cell” as used herein means a battery that generates a current-voltage through utilization of a photovoltaic effect in which light energy is absorbed from sunlight to generate an electron and a hole.

Recently, an n-p diode-type silicon (Si) single crystal-based solar cell having a light energy conversion efficiency of more than 20% has been widely known, and has been actually used in solar power generation. However, the solar cell requires a high temperature treatment step and the price of a material itself is high, and hence there is a problem in that the cost per unit electric power is high. In addition, there is a problem with its supply property in terms of a silicon resource.

Meanwhile, a solar cell using an organic material (hereinafter also referred to as “organic solar cell”) does not require the high temperature treatment step, and can be produced in a so-called roll-to-roll system using a sheet-shaped substrate, and hence a cost reduction can be expected. In particular, the development of a perovskite solar cell including a crystal having a perovskite structure (hereinafter also referred to as “perovskite crystal”) as a photoelectric conversion layer toward its practical use has been advanced because the cell is excellent in photoelectric conversion characteristic.

Various production methods have been proposed in order to form a film of the perovskite crystal. In investigations at a research level, a film formation method using spin coating has been widely used. Meanwhile, in investigations aiming at mass production, a film formation method suitable for mass production, such as die coating or blade coating, has been widely used from the viewpoints of connectivity with preceding and succeeding processes, the size of an area where a film can be formed at one time, and application to the roll-to-roll system.

Meanwhile, the perovskite crystal is highly difficult to produce, and hence defects of various sizes, such as a defect at a molecular level and a defect as a coating film, occur. Any such defect leads to a reduction in carrier transportability to upper and lower layers, that is, a reduction in photoelectric conversion efficiency, and the promotion of the deterioration of the perovskite solar cell due to long-term use. Accordingly, research for suppressing a defect and research for improving the carrier transportability have been actively conducted.

In Japanese Patent Laid-Open No. 2023-148126, there is a description of a technology of applying a material film of a perovskite thin film using a coating method suitable for increasing an area.

In US Patent Application Publication No. 2021/057591, there is a description of a technology including forming an ultrathin insulating material layer, which interacts with a defect, on the surface of a perovskite crystal to protect the defect, to thereby improve the performance of a perovskite solar cell.

In Japanese Patent Laid-Open No. 2018-170382, there is a description of a technology including incorporating an organic semiconductor and a polymer compound having a glass transition temperature of 100° C. or more into a hole-transporting layer to reduce its peeling from an electrode.

In Q. Hu, et al., Sol. RRL, 2019, 3, 1800264, there is a description that conversion efficiency is improved by mixing copper phthalocyanine and a conductive polymer into a hole-transporting layer.

According to investigations made by the inventors, it has been found that there is room for improvements in mass productivity and uniformity of photoelectric conversion efficiency in each of the photoelectric conversion elements described in Japanese Patent Laid-Open No. 2023-148126, US Patent Application Publication No. 2021/057591, Japanese Patent Laid-Open No. 2018-170382, and Q. Hu, et al., Sol. RRL, 2019, 3, 1800264.

### SUMMARY

The present disclosure is directed to providing a method of producing a photoelectric conversion element that achieves both mass productivity and the uniformity of photoelectric conversion efficiency.

The above-mentioned provision is achieved by the present disclosure described below. That is, according to the present disclosure, there is provided a method of producing a photoelectric conversion element, the method comprising: a step (A) of forming a photoelectric conversion layer containing a crystal having a perovskite structure by using a large-area film formation method; and a step (B) of forming a charge-transporting layer by applying a paint for a charge-transporting layer onto a surface of the photoelectric conversion layer and then drying the paint, wherein the paint for a charge-transporting layer contains a charge-transporting particle and an insulating resin.

Features of the present disclosure will become apparent from the following description of embodiments with reference to the attached drawings.

### BRIEF DESCRIPTION OF THE DRAWINGS

FIG. 1 is a schematic view of a layer configuration in the thickness direction of a photoelectric conversion element according to a first embodiment of the present disclosure.

FIG. 2 is a perspective view for schematically illustrating a moving body including the photoelectric conversion element according to one embodiment of the present disclosure.

FIG. 3 is a perspective view for schematically illustrating a building material including the photoelectric conversion element according to one embodiment of the present disclosure.

FIG. 4 is a schematic sectional view of the photoelectric conversion element according to one embodiment of the present disclosure.

### DESCRIPTION OF THE EMBODIMENTS

The present disclosure is described in detail below by way of illustrative embodiments.

### One Embodiment

One embodiment is directed to a method of producing a photoelectric conversion element.

The method of producing a photoelectric conversion element of the present disclosure is a method of producing a photoelectric conversion element comprising: a step (A) of forming a photoelectric conversion layer containing a crystal having a perovskite structure by using a large-area film formation method; and a step (B) of forming a charge-transporting layer by applying a paint for a charge-transporting layer onto a surface of the photoelectric conversion layer and then drying the paint, wherein the paint for a charge-transporting layer contains a charge-transporting particle and an insulating resin.

As a result of investigations, the inventors have found that, when a photoelectric conversion element having a charge-transporting layer is produced through the above-mentioned process, a photoelectric conversion element that achieves both mass productivity and the uniformity of photoelectric conversion efficiency is obtained. The reason for the foregoing is conceived as described below.

FIG. 4 is a schematic sectional view of a photoelectric conversion element according to one embodiment of the present disclosure. A photoelectric conversion element 1 includes, on a substrate 2, a second electrode 3, a photoelectric conversion layer 11, a charge-transporting layer containing a charge-transporting particle 12 and an insulating resin 13, and a first electrode 7. One of the first electrode 7 and the second electrode 3 is an anode, and the other is a cathode. A current can be extracted by connecting the first electrode 7 and the second electrode 3 with an external circuit.

First, mass productivity can be improved by forming the photoelectric conversion layer 11 by using the large-area film formation method. The use of the large-area film formation method enables the production of the many photoelectric conversion elements 1 in a limited time, and hence can reduce production cost. In addition, the connectivity with preceding and succeeding processes in a production line is improved, and the photoelectric conversion layer can be applied to a roll-to-roll system. Unlike the formation of any other layer, the formation of the photoelectric conversion layer 11 requires appropriate production of a perovskite crystal, and thus the photoelectric conversion layer is highly difficult to produce. Accordingly, it is important to improve the mass productivity in the film formation of the photoelectric conversion layer as compared to the formation of any other layer.

In the photoelectric conversion layer 11 formed of the perovskite crystal, unevenness may be present on a crystal surface or a gap may be present between crystal grains. When a defect, such as the unevenness or the gap, is present, the photoelectric conversion efficiency reduces, and the deterioration of the layer is liable to progress owing to oxygen or the like entering the defect. Accordingly, the defect is preferably filled with the charge-transporting layer or the like. However, when a low-resistance charge-transporting layer is formed on the surface of the photoelectric conversion layer containing the crystal having a perovskite structure, the surface up to the side surface of the unevenness or the gap is covered with a low-resistance material with respect to the flow of charge.

As a result, a state in which an electron and a hole easily recombine is established, and the state may lead to a reduction in photoelectric conversion efficiency. In addition, when a high-resistance charge-transporting layer is formed on the surface of the photoelectric conversion layer containing the crystal having a perovskite structure, the exchange of charge is hindered, and hence the conversion efficiency of the photoelectric conversion element may reduce.

Meanwhile, when a charge-transporting layer is formed of the charge-transporting particle 12 and the insulating resin 13 on the perovskite crystal, the charge-transporting particle 12 and the insulating resin 13 are present independently. As a result, the insulating resin 13 preferentially penetrates into the side surfaces of the unevenness and gap of the perovskite crystal, and hence the recombination of an electron and a hole occurring there is suppressed.

At the same time, it is conceived that, when a charge-transporting material is present as a particle on the perovskite crystal, charge transportation efficiency from the perovskite crystal to the first electrode 7 is maintained while the insulating resin 13 is prevented from becoming an obstacle to charge transportation.

Further, as a result of intensive investigations made by the inventors, it has been found that the uneven shape and gap of the photoelectric conversion layer are more liable to occur as the layer is formed by using the large-area film formation method to a larger extent, and an improving effect on the uniformity of the conversion efficiency of the photoelectric conversion element exhibited by forming the above-mentioned charge-transporting layer becomes even higher. Possible reasons why the uneven shape is liable to occur in the photoelectric conversion layer by the large-area film formation method are, for example, the following reasons.

A first possible reason is the formation of a photoelectric conversion layer with a large area. When a photoelectric conversion layer is formed with a large area, the probability that an uneven shape or a gap occurs somewhere increases by the amount of the increased area. In general, when a defect is present in any one location of the photoelectric conversion layer in a photoelectric conversion element, the recombination of carriers and the deterioration of a sample progress therefrom serving as a starting point. Accordingly, the photoelectric conversion efficiency and durability of the photoelectric conversion element reduce more than the area of the defect portion.

A second possible reason is a method of forming the photoelectric conversion layer. In experiments at a research level, a photoelectric conversion layer is often formed by combining an approach including forming a uniform wet film by rotating a substrate (hereinafter also referred to as “spin coating method”) and an approach including growing a crystal having a perovskite structure by applying a poor solvent to the wet film (hereinafter also referred to as “poor solvent method”).

When mass production is assumed, the step of applying the poor solvent has a problem in terms of environmental load, and has a problem in terms of cost because a step of removing the solvent is separately required. Accordingly, a technique of promoting the growth of a crystal having a perovskite structure by blowing a gas or reducing a pressure is often adopted. In each of those techniques, the speed at which crystallization progresses is slower than that in the poor solvent method in which a good solvent in a material is instantly replaced. Accordingly, the size of the crystal to be obtained varies, and hence an uneven shape is liable to occur in the photoelectric conversion layer.

The effects of the present disclosure can be achieved when the respective configurations synergistically exhibit effects on each other as in the mechanism as described above.

The present disclosure is described in detail below by way of illustrative embodiments. The present disclosure is not limited to the following embodiments, and the following embodiments, which are appropriately changed, modified, and the like based on the ordinary knowledge of a person skilled in the art without departing from the gist of the present disclosure, are also encompassed within the scope of the present disclosure.

The term “layer” as used herein means not only a layer having a clear boundary or a layer having a flat thin film shape but also a layer having a concentration gradient in which the concentration of a constituent element gradually changes, or a layer that may form a complicatedly intricate structure together with another layer. In addition, the elemental analysis of the layer may be performed by, for example, performing the TOF-SIMS/FE-TEM/EDS line analysis measurement of a cross section of the photoelectric conversion element and observing the element distribution of a specific element. The analysis of each layer may be performed by peeling and removing a layer from a completed photoelectric conversion element to expose the layer to be analyzed. In the present disclosure, for the quantification of a volume ratio, the area ratio of an exposed surface or a cross section is used as the volume ratio of the layer.

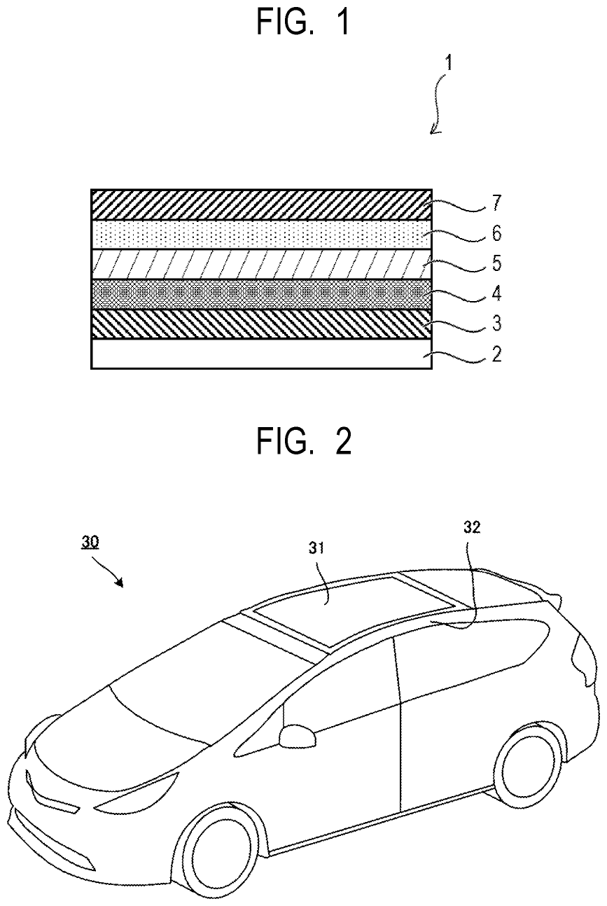

FIG. 1 is a sectional view for schematically illustrating the configuration of the photoelectric conversion element according to one embodiment of the present disclosure. The photoelectric conversion element 1 includes, on the substrate 2, the second electrode 3, an electron-transporting layer 4, a photoelectric conversion layer 5, a charge-transporting layer 6, and the first electrode 7. One of the first electrode 7 and the second electrode 3 is an anode, and the other is a cathode. A current can be extracted by connecting the first electrode 7 and the second electrode 3 with an external circuit.

The photoelectric conversion layer 5 is excited by light that has entered the layer through the substrate 2, the second electrode 3, and the electron-transporting layer 4, or the first electrode 7 and the charge-transporting layer 6 to generate an electron or a hole. That is, the photoelectric conversion layer 5 generates a current between the first electrode 7 and the second electrode 3. The electron-transporting layer 4 is a layer arranged between the photoelectric conversion layer 5, and the two electrodes (the second electrode 3 and the first electrode 7), and may not be formed in some cases. A form in which the plurality of electron-transporting layers 4 and photoelectric conversion layers 5 are laminated may be adopted. Such form may also be referred to as “tandem structure.” The respective members are described below. In addition, the photoelectric conversion element may be produced in the order of the first electrode 7, the charge-transporting layer 6, the photoelectric conversion layer 5, the electron-transporting layer 4, and the second electrode 3 on the substrate 2.

### [Step of Forming Photoelectric Conversion Layer (Step (A))]

The method of producing a photoelectric conversion element of the present disclosure includes the step (A) of forming the photoelectric conversion layer containing the crystal having a perovskite structure by using the large-area film formation method.

The large-area film formation method may be classified into a coating method and a vapor deposition method.

The vapor deposition method is a method of forming a film by volatilizing a solid raw material and solidifying the raw material again on a base material surface. Examples of the vapor deposition method include a physical vapor deposition (PVD) method and a chemical vapor deposition (CVD) method. Examples of the PVD method include a co-evaporation method, a pulsed laser deposition (PLD) method, an electron beam heating vapor deposition method, and a molecular beam epitaxy method. Examples of the CVD method include thermal CVD, plasma CVD, and an atomic layer deposition (ALD) method.

The coating method is a method including preparing a coating liquid for each layer to be described later, applying the coating liquid onto a desired base material or layer, and drying the coating liquid. In the method of producing a photoelectric conversion element of the present disclosure, the step (A) preferably includes: a step (A-1) of uniformly applying a paint for a photoelectric conversion layer; and a step (A-2) of drying the paint for a photoelectric conversion layer. With such configuration, the mass productivity of the method of producing a photoelectric conversion element of the present disclosure can be further improved.

Examples of the step (A-1) include dip coating, spray coating, ink jet coating, screen coating, roll coating, gravure coating, die coating, blade coating, curtain coating, and bar coating. A method is appropriately selected from those methods in accordance with the characteristics of the photoelectric conversion layer to be produced, such as thickness control and orientation control.

The photoelectric conversion layer requires the production of the perovskite crystal in a targeted state in addition to general film formation techniques, and hence its area is highly difficult to increase. The spin coating method requires the rotation of a substrate or film at a high speed owing to its properties, and hence involves a problem in increasing the area from the viewpoints of safety and uniformity. In addition, the spin coating method inevitably becomes batch-type production, and hence involves a problem in its connection with preceding and succeeding processes in a production line. Accordingly, the spin coating method is inappropriate as a large-area film formation method for a photoelectric conversion layer.

The method of the step (A-1) as described above is merely an example, and a coating method obtained by improving those general-purpose coating methods may be used. In addition, the step (A-1) may be used alone or in combination with any other coating method. In the method of producing a photoelectric conversion element of the present disclosure, from the viewpoint that its mass productivity can be further improved, the step (A-1) preferably includes a coating process by any one coating method selected from the group consisting of die coating, blade coating, roll coating, spray coating, ink jet coating, gravure coating and screen coating out of those coating methods, and more preferably includes die coating.

The dip coating is a coating method including immersing a base material in a paint and then pulling up the base material. The spin coating is a method including dropping a paint onto a base material and rotating the base material to generate a centrifugal force, to thereby uniformly form a coating film. The spray coating is a method of forming a coating film by spraying a paint onto a base material surface. The ink jet coating is a method of uniformly forming a coating film by dropping a paint onto a base material surface drop by drop.

The screen coating is a coating method including using a plate using a screen mesh woven from synthetic fibers or metal fibers. A paint passes through the mesh of the screen mesh and is applied to a base material. The roll coating is a coating method including using one or more rollers to provide an optimum coated surface. Application examples of the roll coating are wide and include various methods, such as: an approach including controlling the amount of a paint on a roller in advance by die coating, blade coating, or the like, and applying the paint onto a base material; an approach including controlling the gap of a paint passing through a pair of rollers with the rollers to make a coating film uniform; and an approach represented by a roll-to-roll system, the approach including uniformizing the coated surface of a deformable film such as a film with a roller.

The gravure coating is a coating method including: attaching a paint to a gravure roll having unevenness on its surface; and transferring the paint to a base material. The die coating is a method of forming a coating film by: extruding a paint at a uniform flow rate in a width direction from a slit having a width called a slot die; and forming a liquid pool called a bead between a base material and the slit.

The blade coating is a method of uniformly forming a coating film by passing a paint dropped onto a base material between the base material and a blade. The curtain coating is a method of forming a coating film by: extruding a paint at a uniform flow rate in a width direction from a slit having a width; and directly transferring the paint onto a base material without forming a liquid pool. The bar coating is a method of uniformly forming a coating film by passing a paint dropped onto a base material between bars having forming therein grooves.

The step (A-2) is a step of drying the wet film of the paint for a photoelectric conversion layer obtained in the step (A-1) to produce a film of the perovskite crystal. The step (A-2) is also a step of producing the nucleus of the perovskite crystal and growing the nucleus in addition to the general drying of the wet film.

When the drying is slow, the wet film moves during its drying and hence unevenness is liable to occur in the film. In addition, the number of the nuclei of the perovskite crystals is so small that a space is liable to occur between the perovskite crystals. Accordingly, a drying speed is preferably fast. Meanwhile, when the drying is too fast, many nuclei of the perovskite crystals are produced, and thus each perovskite crystal becomes smaller.

As the perovskite crystal becomes smaller, the number of interfaces between the perovskite crystals increases accordingly. Accordingly, a reduction in photoelectric conversion efficiency of the photoelectric conversion element due to an increase in interface resistance and a reduction in durability thereof due to the raw material ions of the perovskite crystal moving through the interface easily occur. Accordingly, the drying speed in the step (A-2) preferably falls within an appropriate range.

Examples of the step (A-2) include a poor solvent method, a gas quench method, a reduced pressure method, a rapid heating method, and air drying. The poor solvent method is a method of producing a perovskite crystal by applying a poor solvent to a wet film obtained by a coating method to instantly replace a good solvent in the paint for a photoelectric conversion layer. The good solvent is instantly replaced, and hence many nuclei of the perovskite crystals are produced.

Accordingly, a film defect is difficult to produce, film roughness tends to be small, and each crystal tends to be small. The gas quench method is a method including blowing a gas to promote the drying of the wet film, to thereby produce the perovskite crystal. The decomposition of the perovskite crystal is promoted by moisture, and hence the gas to be blown is preferably a low-moisture gas.

Examples of the gas to be blown include a nitrogen gas and dry air. Hot air may be blown by heating the gas in advance. The reduced pressure method is a method including bringing a space where the wet film is present into a reduced pressure state to promote the volatilization of a solvent in the wet film, to thereby dry the wet film.

The rapid heating method is a method of promoting drying by heating the wet film. Examples of the heating include: a method including bringing a heat source into contact with a substrate to directly heat the wet film; and a method including indirectly heating the wet film with infrared rays or the like.

In the method of producing a photoelectric conversion element of the present disclosure, the step (A-2) is preferably free of a process of applying a poor solvent onto a raw material for the crystal having a perovskite structure (free of a poor solvent method). The poor solvent method requires additional use of a solvent that imposes an environmental load, and hence the production method is preferably free of the poor solvent method from the viewpoint of reducing the environmental load and the viewpoint of cost for adding a process of removing the poor solvent.

In the case of a method that does not use the poor solvent method, the rate of the nucleus generation of the perovskite crystal is slower than that in the poor solvent method. Accordingly, a film defect is relatively liable to be produced, film roughness tends to be large, and each crystal tends to be large. In the method of producing a photoelectric conversion element of the present disclosure, the step (A-2) is preferably a process of drying the paint for a photoelectric conversion layer by blowing a gas through use of a unit for blowing a gas (a process involving using a gas quench method). The gas quench method is excellent in cost effectiveness because an apparatus can be assembled relatively easily. In addition, the method enables the drying of the wet film at a desired speed.

In the method of producing a photoelectric conversion element of the present disclosure, in the step (A), the photoelectric conversion layer has a film formation area of preferably 5 cm² or more, more preferably 20 cm² or more. With such configuration, the mass productivity of the method can be further improved, and the recombination-suppressing effect of the insulating resin can be increased. As the film formation area of the photoelectric conversion layer becomes larger, the mass productivity becomes more excellent. In addition, as the film formation area becomes larger, an uneven shape and a void more easily occurs in the photoelectric conversion layer, and the recombination-suppressing effect of the insulating resin is more easily exhibited.

In the film formation of the photoelectric conversion layer in the present disclosure, the raw material for the perovskite crystal may be formed into a film at one time, or the raw material may be formed into a film in a plurality of times. When the raw material is formed into a film in a plurality of times, the effects of the present disclosure are exhibited by including the step (A) in at least one of the divided times.

Annealing treatment may be performed under reduced pressure or under an inert atmosphere (under a nitrogen or argon atmosphere) in order to completely remove a solvent or a dispersion medium from the liquid containing the material for the photoelectric conversion layer. The temperature of the annealing treatment is preferably 40 to 300° C., more preferably 50 to 150° C. The annealing treatment is preferably performed because materials for forming the respective layers may permeate each other at an interface between laminated layers to increase a contact area, and hence a short-circuit current can be increased.

### [Step of Forming Charge-Transporting Layer (Step (B))]

The method of producing a photoelectric conversion element of the present disclosure includes the step (B) of forming the charge-transporting layer by applying the paint for a charge-transporting layer onto the surface of the photoelectric conversion layer and then drying the paint. In the step (B), examples of a method of coating the paint for a charge-transporting layer include dip coating, spin coating, spray coating, ink jet coating, screen coating, roll coating, gravure coating, die coating, blade coating, curtain coating, and bar coating.

Unlike the formation of the photoelectric conversion layer, difficulty in the film formation of the charge-transporting layer reduces as there is no perovskite crystal formation. Accordingly, a spin coating method may also be used. In the method of producing a photoelectric conversion element of the present disclosure, from the viewpoint that its mass productivity can be further improved, the step (B) preferably includes a coating process by any one coating method selected from the group consisting of die coating, blade coating, roll coating, spray coating, ink jet coating, gravure coating and screen coating out of those methods, and more preferably includes a coating process by die coating.

The step (B) may be used alone or in combination with any other coating method. Further, in the method of producing a photoelectric conversion element of the present disclosure, the step (B) preferably includes a coating process by a coating method similar to that in the step (A-1) from the viewpoints of further improving the mass productivity, keeping apparatus cost low, and improving the connectivity of the step with other processes.

A method of drying the paint for a charge-transporting layer is not particularly limited, and examples thereof include heating, gas blowing, and a pressure reduction. Of those, drying by heating is preferred from the viewpoint of simplicity.

In the step (B), a method including applying a resin solution in which the insulating resin is dissolved is preferably used. Thus, the insulating resin preferentially penetrates into a gap between perovskite crystal grains with ease.

In addition, in the step (B), for example, the following methods may each be used: a method including arranging a charge-transporting particle on the surface of the photoelectric conversion layer and then applying the resin solution in which the insulating resin is dissolved; a method including applying the resin solution in which the insulating resin is dissolved on the surface of the photoelectric conversion layer and then arranging the charge-transporting particle; or a method including applying the resin solution in which the insulating resin is dissolved and a solution in which the charge-transporting particle is dispersed on the surface of the photoelectric conversion layer.

### [Charge-Transporting Particle]

In the method of producing a photoelectric conversion element of the present disclosure, the paint for a charge-transporting layer contains the charge-transporting particle. The incorporation of the charge-transporting particle enables a smooth flow of charge generated in the photoelectric conversion layer. The average particle diameter of the charge-transporting particles is preferably 1.0×10¹ to 3.0×10² nm.

When the average particle diameter falls within the above-mentioned range, the penetration of the charge-transporting particle into a gap between the perovskite crystal grains can be suppressed and the uniformity of a film is maintained, and hence the loss of charge transportability can be suppressed. The particle diameter of the charge-transporting particle in the charge-transporting layer may be determined as a volume-average particle diameter from a particle size distribution determined by an image imaging method using a scanning electron microscope (SEM).

A particle having a small aspect ratio, such as a substantially spherical shape, a rugby-ball shape, a cylindrical shape, or a flat shape, is preferred as the particle shape of the charge-transporting particle. For example, when particles each having a large aspect ratio such as needle-shaped particles are adopted, the particles need to be deposited in a unified orientation to improve coverage.

That is, even when the charge-transporting particles can be deposited on the photoelectric conversion layer in a stable dispersed state without causing excessive aggregation, a portion where the photoelectric conversion layer cannot be covered with the charge-transporting particles is liable to remain owing to their shapes. In addition, the particle is liable to stack on any other particle, and hence a void is liable to remain between the photoelectric conversion layer and a layer containing the charge-transporting particle. Thus, the effects of the present application become smaller.

The aspect ratio of the charge-transporting particle is preferably 5 or less, more preferably 3 or less. A particle shape that reduces the degrees of the effects of the present application is, for example, a needle shape or a fibrous shape. The aspect ratio is larger than 10.

The shape and aspect ratio of the charge-transporting particle may be observed with a scanning electron microscope (SEM) or a transmission electron microscope (TEM).

In addition, specific material examples of the charge-transporting particle include a phthalocyanine pigment, an azo pigment, a lake pigment, a quinacridone pigment, a dioxazine pigment, a perylene pigment, and an isoindolinone pigment.

In the method of producing a photoelectric conversion element of the present disclosure, the charge-transporting particle preferably contains a cyclic conjugated compound in which a plurality of pyrrole rings are bonded by conjugated bonds. When the cyclic conjugated compound in which the plurality of pyrrole rings are bonded by covalent bonds is formed into the charge-transporting particle, high charge transportability is exhibited.

In the method of producing a photoelectric conversion element of the present disclosure, the charge-transporting particle preferably contains a phthalocyanine compound, more preferably contains a structure represented by the following formula (Pc-2), and still more preferably contains a hydroxygallium phthalocyanine compound. The charge-transporting particle can more efficiently transport charge generated in the photoelectric conversion layer.

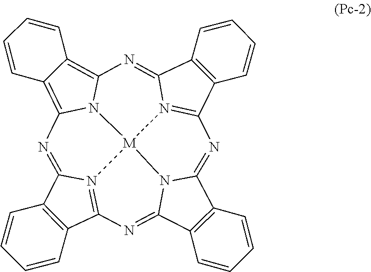

M in the formula (Pc-2) represents H₂, a metal atom having a ligand, or a metal atom free of a ligand.

The structure of a chemical substance such as the charge-transporting particle of the present disclosure may be identified by a nuclear magnetic resonance (NMR) method.

In particular, when M in the formula (Pc-2) represents H₂, the formula (Pc-2) is represented by the following formula (Pc-1).

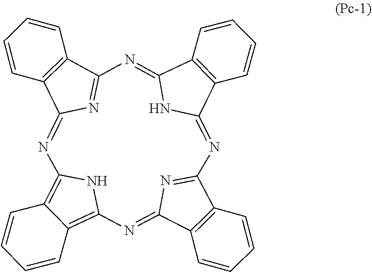 [SMILES: c1ccc2c(c1)-c1nc-2nc2[nH]c(nc3nc(nc4[nH]c(n1)c1ccccc41)-c1ccccc1-3)c1ccccc21]

### [Insulating Resin]

In the method of producing a photoelectric conversion element of the present disclosure, the paint for a charge-transporting layer contains the insulating resin. Specific examples of the insulating resin include a polyacetal resin, an acrylic resin, a polyarylate resin, a polycarbonate resin, a polyvinyl acetate resin, a polyester resin, a polyamide resin, a polyurethane resin, and a polystyrene resin. In the method of producing a photoelectric conversion element of the present disclosure, the insulating resin is preferably a polyvinyl acetal resin or a polyvinyl butyral resin out of those resins. The insulating resin is easily brought into close contact with a charge-transporting material (charge-transporting particle), and hence a more effective charge distribution can be formed.

In the method of producing a photoelectric conversion element of the present disclosure, the glass transition temperature of the insulating resin is preferably less than 100° C., more preferably 95° C. or less. When the glass transition temperature falls within the range, the insulating resin is easily brought into close contact with the charge-transporting material (charge-transporting particle), and hence a more effective charge distribution can be formed. The glass transition temperature may be determined with a differential scanning calorimeter (DSC).

In the present disclosure, the molecular weight of the insulating resin is preferably 10,000 or more.

In the present disclosure, it is preferred that the charge-transporting layer contain an aromatic ring compound having a hydroxy group, the aromatic ring compound being different from the charge-transporting material (charge-transporting particle) and the insulating resin. When the charge-transporting layer contains the aromatic ring compound having a hydroxy group, the charge-transporting material (charge-transporting particle) and the insulating resin are more easily brought into contact with each other, and hence a more effective charge distribution can be formed.

In the method of producing a photoelectric conversion element of the present disclosure, the photoelectric conversion element may include, between its first electrode and charge-transporting layer, a second charge-transporting layer free of a charge-transporting particle on the charge-transporting layer. With such configuration, the delivery of a carrier to an electrode may be facilitated.

The respective layers are described below.

### [Photoelectric Conversion Element]

The photoelectric conversion element of the present disclosure is characterized by including: the photoelectric conversion layer containing the crystal having a perovskite structure; and the charge-transporting layer containing the charge-transporting particle and the insulating resin. The photoelectric conversion element may include: the first electrode; the second electrode; the photoelectric conversion layer arranged between the first electrode and the second electrode, the photoelectric conversion layer containing the crystal having a perovskite structure; and the charge-transporting layer between the photoelectric conversion layer and the first electrode.

In addition, in order to improve the photoelectric conversion efficiency, a tandem type in which the photoelectric conversion elements are laminated may be adopted. The kind of the photoelectric conversion element to be laminated is not limited, and for example, a silicon solar cell or a CIGS solar cell may be adopted in addition to a perovskite solar cell using a perovskite crystal in its photoelectric conversion layer.

A method of forming each of the layers including the photoelectric conversion layer and charge-transporting layer of the photoelectric conversion element of the present disclosure is, for example, a coating method or a vapor deposition method. Examples of the coating method include dip coating, spin coating, spray coating, ink jet coating, meniscus coating, screen coating, roll coating, die coating, blade coating, curtain coating, and wire bar coating. A desired method may be selected as such forming method in accordance with each layer.

The respective layers are described below.

### [Substrate]

The photoelectric conversion element 1 of the present disclosure may include the substrate 2, and examples thereof include a transparent glass substrate made of soda-lime glass or alkali-free glass, a ceramic substrate, and a transparent plastic substrate. When light is taken in from the first electrode 7 side, an opaque material may be used as the substrate 2, and when light is taken in from the second electrode 3 side, the substrate 2 is formed of a transparent material.

### [Electrode]

The photoelectric conversion element of the present disclosure includes the first electrode and the second electrode. A material for the first electrode 7 or the second electrode 3 is not particularly limited, and a material that has hitherto been known may be used. Examples thereof include: metals, such as gold, silver, titanium, and copper; sodium; a sodium-potassium alloy; lithium; magnesium; carbon; a carbon nanotube; aluminum; a magnesium-silver mixture; a magnesium-indium mixture; an aluminum-lithium alloy; an Al/Al₂O₃ mixture; and an Al/LiF mixture.

Examples of a transparent electrode material include: conductive transparent materials, such as CuI, indium tin oxide (ITO), SnO₂, aluminum zinc oxide (AZO), indium zinc oxide (IZO), gallium zinc oxide (GZO), fluorine-doped tin oxide (FTO), and antimony-doped tin oxide (ATO); and conductive transparent polymers. Those materials may be used alone or in combination thereof.

At least one electrode of the first electrode 7 or the second electrode 3 on a light incident side is a transparent electrode, and the other may be a transparent electrode or may also serve as a reflective layer formed of a light reflective material, or may be a transparent electrode including a reflective layer on a side opposite to the light incident side. When the first electrode 7 is on the light incident side, the second electrode 3 and the substrate 2 may be a transparent electrode and a reflective layer, respectively. The electrode may be a patterned electrode.

### [Step of Forming First Electrode and Step of Forming Second Electrode]

The method of producing a photoelectric conversion element of the present disclosure includes the steps of: forming the first electrode; and forming the second electrode. In the step of forming the first electrode and the step of forming the second electrode, an appropriate method may be selected in accordance with a material for the first electrode and a material for the second electrode, respectively. Examples of such method include, but are not limited to, a sputtering method, a vacuum vapor deposition method, a vapor phase growth method (CVD method), and a spray pyrolysis deposition method (SPD method). The materials for the first electrode and the second electrode are as described above. When one, or each of both, of the first electrode and the second electrode is a transparent electrode, the thickness of the transparent electrode is preferably 0.03 to 3 μm.

When a solar cell is produced, cutting processing is generally performed for circuit formation between steps. Examples of the cutting processing include mechanical patterning and laser patterning.

### [Photoelectric Conversion Layer]

The photoelectric conversion element of the present disclosure includes the photoelectric conversion layer containing the crystal having a perovskite structure. The photoelectric conversion layer 5 contains the crystal having a perovskite structure. The crystal having a perovskite structure to be used in the present disclosure is preferably represented by the following general formula [1].

ABX₃ [1]

In the general formula [1], A represents a monovalent cation of an organic molecule or a metal atom, B represents a divalent metal cation, and X represents a monovalent halide anion.

A in the general formula [1] preferably represents CₚNₘHₙ (“p”, “m”, and “n” each represent a positive integer) in the case of, for example, the organic molecule. Specific examples thereof include methylammonium and formamidinium.

In addition, the metal atom is not particularly limited, and lithium, cesium, sodium, potassium, and rubidium are preferred. Those organic molecules or metal atoms may be used alone or in combination thereof.

When the cation A to be included is too large to fit in a crystal having a three-dimensional perovskite structure, a crystal having a two-dimensional perovskite structure, a crystal having a 2.5-dimensional perovskite structure with properties of both the two-dimensional and three-dimensional perovskite structures, a two-layer crystal having three-dimensional and two-dimensional perovskite structures, or a crystal having a mixed three-dimensional/two-dimensional perovskite structure is formed, and any of the structures functions as the photoelectric conversion layer.

The two-layer crystal having three-dimensional and two-dimensional perovskite structures refers to a crystal in which the crystals having three-dimensional and two-dimensional perovskite structures are layered as independent and separate layers. The crystal having a mixed three-dimensional/two-dimensional perovskite structure refers to a crystal having a structure in which both the regions or domains of crystals having two-dimensional or 2.5-dimensional layered and three-dimensional perovskite structures are mixed.

It is preferred that the crystal having a two-dimensional perovskite or 2.5-dimensional perovskite structure be represented by each of the following general formulae [2] to [4]. “n” represented in each of the following general formulae represents a positive integer.

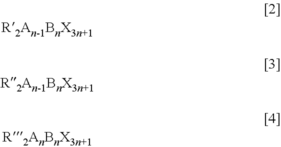

The general formula [2], the general formula [3], and the general formula [4] form perovskite structures of a Ruddlesden-Popper (RP) type, a Dion-Jacobson (DJ) type, and an Alternating cations in the interlayer (ACI) type, respectively.

R′, R″, and R′″ in the general formulae [2] to [4] each represent a cation of an organic molecule, which may have a substitute, or of a metal. Specifically, ethylammonium, propylammonium, n-butylammonium, n-hexylammonium, n-octylammonium, 1,6-hexanediammonium, iso-butylammonium, 3-(nonafluoro-tert-butyloxy)propylamine, 1,3-propanediammonium, 1,5-pentamethylenediamine, octyldiammonium, 2,2-(ethylenedioxy)bis(ethylammonium), 5-aminovaleric acid, 4-tert-butylammonium, N,N′-dimethylethylene-1,2-diammonium, 2,2,3,3,3-pentafluoropropylammonium, guanidinium, propylammonium, propargylamine, an alkylammonium, cyclohexylmethylammonium, 4-(aminomethyl)piperidinium, piperidinium, pyrrolidinium, cyclohexylammonium, 4-fluorophenethylammonium, trifluoromethylbenzylammonium, pentafluorobenzylammonium, pentafluorophenylethylammonium, 4-methoxyphenethylammonium, imidazolium, pyridinium, 3-thiophenemethylammonium, 2-thiopheneethylammonium, 2-thiopheneformamidinium, 2-thiophenemethylammonium, 1-naphthylmethylammonium, 2-naphthylmethylammonium, phenethylammonium, phenylammonium, benzylammonium, 2,5-thiophenedimethylammonium, phenylpropylammonium, 1,4-phenylenedimethanamine, 3-phenyl-2-propen-1-ammonium, phenylbutylammonium, 4-tert-butylbenzylammonium, and 3-(aminomethyl)piperidinium are preferred.

B in each of the general formulae [1] to [4] represents a metal atom, and examples thereof include lead, tin, bismuth, zinc, titanium, antimony, nickel, iron, cobalt, silver, copper, gallium, germanium, magnesium, calcium, indium, aluminum, manganese, chromium, molybdenum, and europium. Of those, lead, tin, and bismuth are preferred from the viewpoint of the overlap of electron orbits. Those metal atoms may be used alone or in combination thereof.

X in each of the general formulae [1] to [4] represents a halogen atom, and examples thereof include chlorine, bromine, and iodine. Those halogen atoms may be used alone or in combination thereof. Of those, a halogen atom is preferred because, when the halogen atom is incorporated into the perovskite structure, the above-mentioned crystal having a perovskite structure easily becomes soluble in an organic solvent, and hence the application to an inexpensive printing method or the like is enabled. Further, iodine is more preferred because the energy bandgap of the crystal having a perovskite structure narrows.

Specifically, as three-dimensional perovskite, two-dimensional perovskite, and mixed three-dimensional/two-dimensional perovskite, MAPbI₃, FAPbCl₃, FAPbI₃, MAPbIₓBr₃₋ₓ, MAPbIₓCl₃₋ₓ, Cs_{0.05}(MA_{0.17}FA_{0.83})_{0.95}Pb(I_{0.83}Br_{0.17})₃, {Csₓ₁(FAₓ₂MA₁₋ₓ₂)₁₋ₓ₁}ₓ₃Pb(Iₓ₄Br₁₋ₓ₄)ₓ₅, Cs_{0.05}FA_{0.88}MA_{0.07}PbI_{2.56}Br_{0.44}, (FAPbI₃)_{0.95}(MAPbBr₃)_{0.05}, (FAPbI₃)_{0.85}(MAPbBr₃)_{0.15}, CsPbI₃, CsPbBr₃, Csₓ(MA)₁₋ₓPbI₃, Csₓ(FA)₁₋ₓPbI₃, MAₓ(FA)₁₋ₓPbI₃, MA_{0.17}FA_{0.83}Pb(I_{0.83}Br_{0.17})₃, Cs_{0.15}FA_{0.85}PbI_{2.55}Br_{0.45}, Cs_{0.05}FA_{0.88}MA_{0.07}PbI_{2.56}Br_{0.44}, Cs_{0.15}FA_{0.85}PbI_{2.55}Br_{0.45}, (PEA)₂(MA)₂Pb₃I₁₀, (PTA)₂(MA)₄Pb₅I₁₆, (PEA)₂(MA)₄Pb₅I₁₆, (ThMA)₂(MA)₂Pb₃I₁₀, (3BBA)₂(MA)₂Pb₃I₁₀, (ThMA)₂(FA)₄Pb₅I₁₆, (4FPEA)₂(FA_{0.3}MA_{0.7})₄Pb₅I₁₆, (PDMA)FA₂Pb₃I₁₀, (3AMPY)(MA)₃Pb₄I₁₃, (PDMA)MA₅Pb₆I₁₉, (PDMA)MA₃Pb₄I₁₃, (TTDMA)MA₃Pb₄I₁₃, (TTDMA)MA₄Pb₅I₁₆, (BA_{0.9}PEA_{0.1})₂MA₄Pb₅I₁₆, (BA_{0.9}PEA_{0.1})₂MA₃Pb₄I₁₃, (4FPEA)₂MA₃Pb₄I₁₃, (4FPEA)₂MA₄Pb₅I₁₆, (BA)₂MA₂Pb₃I₁₀, (BA)₂MA₃Pb₄I₁₃, (TEA)₂MA₂Pb₃I₁₀, (BA)₂MA₄Pb₅I₁₆, (BA)₂MA₃Pb₄I₁₃, CsSnBr₃, CsSnI₃, FA_{0.75}MA_{0.25}Sn_{0.95}Ge_{0.05}I₃, FAMASnGeI₃, FASnBr₃, FASnI₃, MA₂Sn₃I₈, MASnBr₃, MASnGeI₃, and MASnI₃ are preferred.

The A site, B site, or X site of each of the general formulae may be adjusted to be deficient or excessive in accordance with purposes, and the combinations of x1 to x5 may be changed in accordance with purposes. The combinations of x1 to x5 are, for example, as shown in Table 1. Particularly preferred ranges are 0.03≤x1≤0.10, 0.80≤x2≤0.96, 0.95≤x3≤1.05, 0.80≤x4≤0.96, and 2.95≤x5≤3.05. MACI may be included as a material for forming a perovskite crystal.

TABLE 1

| x1 | x2 | 1-x2 | x3 | x4 | 1-x4 | x5 |
|---|---|---|---|---|---|---|
| 0.05 | 0.83 | 0.17 | 1.00 | 0.83 | 0.17 | 3.00 |
| 0.05 | 0.83 | 0.17 | 0.99 | 0.83 | 0.17 | 2.99 |
| 0.05 | 0.83 | 0.17 | 0.98 | 0.83 | 0.17 | 2.98 |
| 0.05 | 0.83 | 0.17 | 0.97 | 0.83 | 0.17 | 2.97 |
| 0.05 | 0.83 | 0.17 | 0.96 | 0.83 | 0.17 | 2.96 |
| 0.05 | 0.83 | 0.17 | 1.01 | 0.83 | 0.17 | 3.01 |
| 0.05 | 0.83 | 0.17 | 1.02 | 0.83 | 0.17 | 3.02 |
| 0.05 | 0.83 | 0.17 | 1.03 | 0.83 | 0.17 | 3.03 |
| 0.05 | 0.83 | 0.17 | 1.04 | 0.83 | 0.17 | 3.04 |
| 0.05 | 0.83 | 0.17 | 1.00 | 0.95 | 0.05 | 3.00 |
| 0.05 | 0.83 | 0.17 | 0.97 | 0.95 | 0.05 | 2.97 |
| 0.05 | 0.83 | 0.17 | 0.98 | 0.95 | 0.05 | 2.98 |
| 0.05 | 0.83 | 0.17 | 0.99 | 0.95 | 0.05 | 2.99 |
| 0.05 | 0.83 | 0.17 | 1.01 | 0.95 | 0.05 | 3.01 |
| 0.05 | 0.83 | 0.17 | 1.02 | 0.95 | 0.05 | 3.02 |
| 0.05 | 0.83 | 0.17 | 1.03 | 0.95 | 0.05 | 3.03 |

In the above-mentioned specific examples, “MA” represents methylammonium, “FA” represents formamidinium, “PEA” represents phenethyl ammonium, “PTA” represents phenyltriethylammonium, “ThMA” represents 2-thiophenemethylammonium, “3BBA” represents 3-bromobenzylammonium, “3AMPY” represents 3-(aminomethyl)pyri dine, “PDMA” represents 1,4-phenylenedimethanammonium, “TTDMA” represents thieno[3,2-b]thiophene-2,5-diyldimethanammonium, “4FPEA” represents 4-fluorophenethylammonium, “BA” represents butylammonium, and “TEA” represents 2-thiopheneethylammonium.

The above-mentioned crystal having a perovskite structure preferably has a cubic structure in which the metal atom B, the organic molecules A, and the halogen atom X are arranged on a body-centered position, the respective corners, and a face-centered position, respectively. The details are not clear, but it is assumed that, when such structure is present, the orientation of an octahedron in a crystal lattice can be easily changed, and hence the mobility of an electron in the crystal having a perovskite structure increases, and the photoelectric conversion efficiency of the photoelectric conversion element is improved.

An organic-inorganic perovskite compound to be used in the present disclosure is preferably a crystalline semiconductor. The term “crystalline semiconductor” means a semiconductor that enables the measurement of an X-ray scattering intensity distribution to detect a scattering peak. When the organic-inorganic perovskite compound is the crystalline semiconductor, the mobility of the electron in the organic-inorganic perovskite compound increases, and the photoelectric conversion efficiency of the photoelectric conversion element is improved.

In addition, the photoelectric conversion layer according to the present disclosure may include a material except the crystal having an organic-inorganic perovskite structure to the extent that the photoelectric conversion efficiency and the charge transportability are not impaired.

The thickness of the photoelectric conversion layer according to the present disclosure is preferably 5 to 2,000 nm. When the thickness is 5 nm or more, light can be sufficiently absorbed, and when the thickness is 2,000 nm or less, the generated charge can be transported to the respective electrodes. A more preferred lower limit is 50 nm, a more preferred upper limit is 1,200 nm, a still more preferred lower limit is 100 nm, and a still more preferred upper limit is 1,000 nm.

In the method of producing a photoelectric conversion element of the present disclosure, the photoelectric conversion layer preferably has a void. In a mass-producible method of producing a photoelectric conversion layer, a void is liable to occur for the reasons described above. In addition, when the photoelectric conversion layer has a void, the charge-transporting particle is liable to penetrate to the second electrode side of the photoelectric conversion layer, and the recombination-suppressing effect of the insulating resin may reduce.

In the method of producing a photoelectric conversion element of the present disclosure, the crystal size of the perovskite structure in the photoelectric conversion layer is preferably 1.0 μm or more. Herein, the crystal size means the major axis of the largest perovskite crystal of observed crystals. The perovskite crystal is large, and hence an uneven shape easily occurs on a perovskite surface and its arithmetic average roughness increases. In the method of producing a photoelectric conversion element of the present disclosure, the arithmetic average roughness Ra of the surface of the photoelectric conversion layer is preferably 10 to 200 nm. When the arithmetic average roughness Ra is 10 nm or more, the recombination-suppressing effect of the insulating resin is more easily exhibited.

In addition, when the arithmetic average roughness Ra is larger than 200 nm, the charge-transporting particle is liable to penetrate to the second electrode side of the photoelectric conversion layer, and the recombination-suppressing effect may reduce. In the method of producing a photoelectric conversion element of the present disclosure, the arithmetic average roughness Ra of the surface of the photoelectric conversion layer is more preferably 20 to 180 nm, still more preferably 40 to 150 nm. In a mass-producible method of producing a photoelectric conversion layer, the perovskite crystal is liable to become larger and the arithmetic average roughness is liable to increase for the reasons described above.

### [Charge-Transporting Layer]

In the photoelectric conversion element of the present disclosure, the charge-transporting layer is arranged between the photoelectric conversion layer and the first electrode, and the charge-transporting layer is formed of the charge-transporting particle and the insulating resin on the surface of the photoelectric conversion layer. In the photoelectric conversion element of the present disclosure, the insulating resin is preferably arranged between the crystals each having a perovskite structure of the photoelectric conversion layer. Each item such as the charge-transporting particle or the insulating resin is as described above.

In the present disclosure, the charge-transporting layer contains the charge-transporting particle serving as a P-type semiconductor and the insulating resin, and the value of the ratio of the volume of the charge-transporting particle in the charge-transporting layer to the volume of the insulating resin in the charge-transporting layer is preferably 5 to 30. The value of the ratio of the volume of the charge-transporting particle to the volume of the insulating resin may be obtained from, for example, the cross-sectional observation of the charge-transporting layer with a scanning electron microscope (SEM), or may be calculated from the weights and specific gravities of the charge-transporting particle and the insulating resin added to the paint for a charge-transporting layer. The insulating resin has a volume resistivity of 10⁸ Ω ·cm or more.

In the method of producing a photoelectric conversion element of the present disclosure, the average thickness of the charge-transporting layer is preferably 1 to 1,000 nm, more preferably 5 to 500 nm, still more preferably 10 to 200 nm, particularly preferably 50 to 200 nm. As the thickness of the charge-transporting layer is larger, a covering effect on a void produced in the photoelectric conversion layer is larger, and an improvement in conversion efficiency when the photoelectric conversion layer is produced by using the large-area film formation method can be expected. In addition, when the thickness of the charge-transporting layer is small, reductions in current and power generation amount due to the charge-transporting layer acting as a resistance can be suppressed.

The charge-transporting layer may be formed by preparing a coating liquid for a charge-transporting layer containing the above-mentioned respective materials and a solvent, forming a coating film of the liquid on the photoelectric conversion layer, and drying the coating film. Examples of the solvent to be used for the coating liquid include an alcohol-based solvent, a ketone-based solvent, an ether-based solvent, an ester-based solvent, and an aromatic hydrocarbon-based solvent. Of those solvents, an alcohol-based solvent or an aromatic hydrocarbon-based solvent is preferred.

### [Second Charge-Transporting Layer]

In the present disclosure, the photoelectric conversion element preferably further includes, between the charge-transporting layer 6 and the first electrode 7, a second charge-transporting layer free of a charge-transporting particle from the viewpoint of the compatibility of a film of the charge-transporting layer 6.

A material for the second charge-transporting layer is, for example, a spirofluorene compound, a triphenylamine compound, a chrysene compound, a pyrene compound, a phthalocyanine compound, a carbazole compound, a fluorene compound, a phenylcyclohexane compound, a benzidine compound, a phenoxazine compound, a phenylenediamine compound, a thiocyanate compound, or a thiophene compound. The compound preferably has an aromatic ring from the viewpoint of the compatibility of a film interface, and Spiro-OMeTAD, PTAA, or a phthalocyanine compound is particularly preferred.

In addition, the second charge-transporting layer may contain a dopant as an additive in order to improve its charge transportation capability. Examples of a substance that may be used as the dopant include lithium compounds such as lithium bis(trifluoromethanesulfonyl)imide, cobalt compounds such as [tris(2-(1H-pyrazol-1-yl)-4-tert-butylpyridine)cobalt(III) tris(bis(trifluoromethylsulfonyl)imide)], boron compounds such as tetrakis(pentafluorophenyl)borate, molybdenum compounds such as tris[1-(methoxycarbonyl)-2-(trifluoromethyl)-ethane-1,2-dithiolene]molybdenum, organic compounds each having a tetracyanoquinodimethane skeleton such as 2,3,4,6-tetrafluoro-7,7,8,8-tetracyanoquinodimethane, and organic compounds each having a pyridine skeleton such as 4-tert-butylpyridine.

### [Electron-Transporting Layer]

In the photoelectric conversion element of the present disclosure, the electron-transporting layer 4 may be arranged between the second electrode 3 and the photoelectric conversion layer 5 as illustrated in FIG. 1.

A material for the electron-transporting layer 4 is not particularly limited, and examples thereof include an N-type conductive polymer, an N-type low-molecular-weight organic semiconductor, an N-type metal oxide, an N-type metal sulfide, a halogenated alkali metal, an alkali metal, and a surfactant. Specific examples thereof include a cyano group-containing polyphenylene vinylene, a boron-containing polymer, bathocuproine, bathophenanthroline, hydroxyquinolinatoaluminum, an oxadiazole compound, a benzimidazole compound, a naphthalenetetracarboxylic acid compound, a fullerene compound, a perylene derivative, a phosphine oxide compound, a phosphine sulfide compound, a fluoro group-containing phthalocyanine, titanium oxide, zinc oxide, indium oxide, tin oxide, gallium oxide, tin sulfide, indium sulfide, and zinc sulfide. In particular, tin oxide may be obtained through the reaction of tin(II) chloride, tin(IV) chloride, tin(II) chloride dihydrate, or tin(IV) chloride pentahydrate.

A preferred lower limit of the thickness of the electron-transporting layer 4 is 1 nm, and a preferred upper limit thereof is 2,000 nm. When the thickness of the electron-transporting layer 4 is 1 nm or more, a hole can be sufficiently blocked, and when the thickness is 2,000 nm or less, the electron-transporting layer 4 is less liable to serve as a resistance at the time of the electron transportation, and hence the photoelectric conversion efficiency is improved. Amore preferred lower limit of the thickness is 3 nm, a more preferred upper limit thereof is 1,000 nm, a still more preferred lower limit thereof is 5 nm, and a still more preferred upper limit thereof is 500 nm.

### [Control of Particle Diameter of Charge-Transporting Particle]

The particle diameter of the charge-transporting particle may be changed by dispersing the coating liquid for a charge-transporting layer with a paint shaker, and the particle diameter may be reduced by increasing a dispersion time. In addition, the particle diameter may be reduced by further subjecting the coating liquid for a charge-transporting layer to a centrifuge.

### [Modularization Step]

The method of producing a photoelectric conversion element of the present disclosure may include a modularization step of sealing the element formed up to the electrode. A method for the sealing is, for example, sealing with a resin or sealing with a film. Examples of a material used for the sealing include a silazane, a silicone rubber, a resin having a siloxane skeleton, and glass.

In addition, hairline treatment may be performed on the surface of the sealed element from the viewpoint of the suppression of adhesion between elements occurring during winding in a roll-to-roll system.

### Application Examples

Application examples of the present disclosure are directed to a photoelectric conversion apparatus, a moving body, and a building material.

### [Photoelectric Conversion Apparatus]

A photoelectric conversion apparatus of the present disclosure includes the above-mentioned photoelectric conversion element. The photoelectric conversion apparatus may be formed by using the plurality of photoelectric conversion elements of the present disclosure. When the plurality of photoelectric conversion elements are connected, such photoelectric conversion apparatus may also be referred to as “photoelectric conversion cell” or “photoelectric conversion module.” Photoelectric conversion elements having different absorption wavelengths may be laminated as the photoelectric conversion elements to increase an output voltage.

In addition, the photoelectric conversion apparatus includes the photoelectric conversion element of the present disclosure and an inverter. The inverter may be a converter for converting a DC voltage to an AC voltage. The photoelectric conversion apparatus may include an electricity storage unit connected to the photoelectric conversion element. The electricity storage unit is not limited as long as the electricity storage unit can store electricity. Examples thereof include a secondary battery using lithium ions, an all-solid-state battery, and an electric double layer capacitor. In order to impart a function of, for example, maintaining or increasing the amount of incident light, a surface layer to which water or dirt is hard to adhere, or a function of collecting or guiding light may be added.

### [Moving Body]

A moving body of the present disclosure includes the above-mentioned photoelectric conversion element. FIG. 2 is a perspective view for schematically illustrating a moving body including the photoelectric conversion element according to one embodiment of the present disclosure. A moving body 30 includes a photoelectric conversion element 31 of the present disclosure and a body 32 including the photoelectric conversion element 31. The photoelectric conversion element 31 is arranged on the position of the body 32 at which ambient light can be received. When the moving body 30 is an automobile, the photoelectric conversion element 31 may be arranged on a roof. Electric energy obtained by the photoelectric conversion element 31 may serve as the power of the moving body 30 or the power of any other electric equipment. Electric energy generated from the power of the moving body 30 may be used for the power of the photoelectric conversion element 31. When the moving body 30 is an automobile, friction energy generated with a brake may be converted into electric energy to be used for the control of the photoelectric conversion element 31.

The moving body 30 may be, for example, an automobile, a motorcycle, a railway vehicle, a ship, or a flying body including an artificial satellite, an airplane, and a drone. The configuration of the body 32 of the moving body 30 is not particularly limited, but is preferably formed of a material having high strength.

### [Building Material]

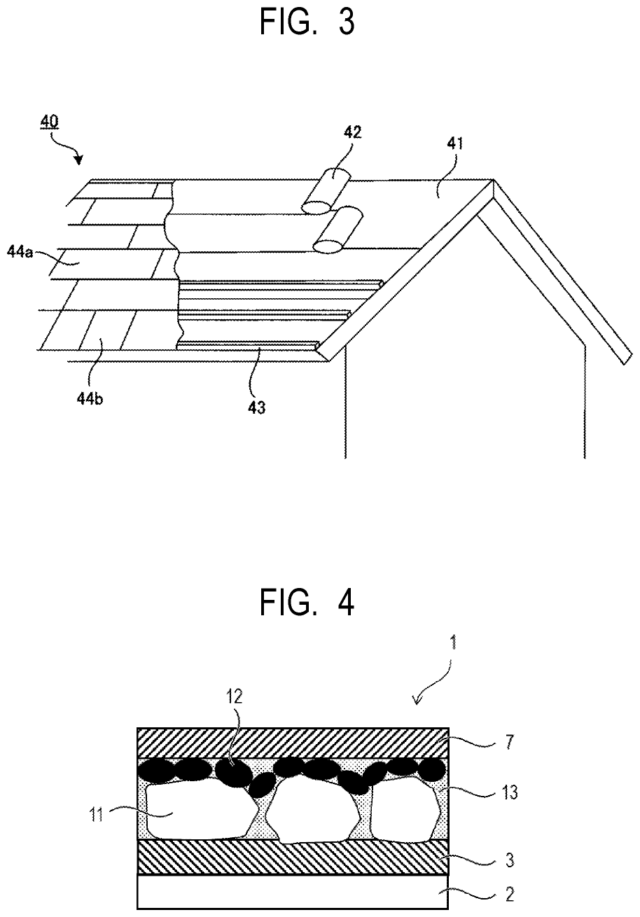

A building material of the present disclosure includes the above-mentioned photoelectric conversion element. FIG. 3 is a perspective view for schematically illustrating a building material including the photoelectric conversion element according to one embodiment of the present disclosure. A building material 40 may be a roof of a building. The building material 40 of this embodiment includes a photoelectric conversion element 41 of the present disclosure, a protective member 42 for protecting the photoelectric conversion element 41, a heat dissipation member 43, and exteriors 44a and 44b.

The building material 40 of the present disclosure may include the heat dissipation member 43 having a thermal conductivity higher than that of the photoelectric conversion element 41. When the building material 40 is used for a roof or the like, the temperature of the photoelectric conversion element 41 may be increased by sunlight, and hence the photoelectric conversion efficiency may be reduced. The reduction of the photoelectric conversion efficiency can be suppressed by using the heat dissipation member 43. Examples of the heat dissipation member 43 include a metal, an alloy, a liquid metal, and a liquid resin.

In addition, the building material 40 of the present disclosure may include the exteriors 44a and 44b. The exterior 44a and the exterior 44b may show different colors, or may show the same color. The exterior 44a and the exterior 44b may be formed of the same member, or may be formed of different members. A paint or a transparent substrate may be used as each of the exteriors. An exterior having small light absorption and a high heat-shielding property is preferred.

In addition to the application examples described above, the following application examples are given: portable devices, such as a calculator, a sensor, and a small solar panel; wearable devices, such as a glasses-type terminal, a watch-type terminal, and a portable medical device; sheet structures supported by a plurality of frames, such as a tent, a plastic greenhouse, and a loading platform of a truck; and structures to be used by being fixed, such as a road surface panel, a floating panel, a building material utilizing the flexibility of a substrate, a wall-type building material, a glass-type building material, and a mega solar panel.

### [Observation of Film Formation Area of Photoelectric Conversion Layer]

The film formation area of the photoelectric conversion layer may be measured by, for example, obtaining an image of a sample having formed thereon the photoelectric conversion layer and performing image processing. In addition, when the formed shape is a general-purpose shape, such as a circle or a rectangle, the film formation area may be calculated by measuring the length of each side of the sample having formed thereon the photoelectric conversion layer with a ruler.

The perovskite crystal has a color such as black, and hence a location where the photoelectric conversion layer is formed can be visually observed. In addition, the perovskite crystal can be distinguished from other layers based on a difference in contrast in an observed image in observation with a scanning electron microscope to be described later or composition analysis by a SEM-EDX function.

In the present disclosure, the photoelectric conversion layer was formed in a rectangular shape. The photoelectric conversion layer of the present disclosure was black, and hence a portion where the photoelectric conversion layer was formed was visually observed. The length of each side of the resultant photoelectric conversion layer was measured with a ruler, and the film formation area was calculated.

### [Observation of Void in Photoelectric Conversion Layer]

Avoid in the photoelectric conversion layer may be observed with a scanning electron microscope (hereinafter also referred to as “SEM”). For example, a void may be observed by performing the surface observation of a sample formed up to the photoelectric conversion layer or performing cross-sectional observation through the cutting of the photoelectric conversion element.

In the present disclosure, the presence or absence of a void in the photoelectric conversion layer was recognized by cross-sectional observation with a SEM (apparatus: SmartSEM manufactured by Carl Zeiss Co., Ltd.) after the photoelectric conversion element had been cut and fixed to an inclined sample stage. In the observation, the charge-transporting layer was distinguished from other layers based on a difference in contrast in an observed image and composition analysis by a SEM-EDX function, and the number of voids was observed in an image taken at a magnification of 10,000. Such image was taken at ten random locations, and the total number of observed voids was counted.

### [Measurement of Average Thickness]

The average thickness of the photoelectric conversion layer or the charge-transporting layer may be measured by, for example, cutting the photoelectric conversion element and performing cross-sectional observation with a SEM.

In the present disclosure, the average thickness of the photoelectric conversion layer or the charge-transporting layer was recognized by cross-sectional observation with a SEM (apparatus: SmartSEM manufactured by Carl Zeiss Co., Ltd.) after the photoelectric conversion element had been cut and fixed to an inclined sample stage. In the observation, the charge-transporting layer was distinguished from other layers based on a difference in contrast in an observed image and composition analysis by a SEM-EDX function, and the average value of the thicknesses of the charge-transporting layer portion was measured by the image processing of an image taken at a magnification of 50,000. Such image was taken at five random locations, and the average of the five average values was adopted as the average thickness of the charge-transporting layer.

### [Measurement of Arithmetic Average Roughness Ra of Photoelectric Conversion Layer]

The arithmetic average roughness Ra of the photoelectric conversion layer is the roughness of the surface on the first electrode side. A measurement method therefor is, for example, a method of subjecting the photoelectric conversion layer after the removal of the first electrode and the charge-transporting layer from the photoelectric conversion element to measurement with an AFM/SPM, or a method of calculating the arithmetic average roughness from a cross-sectional image of the photoelectric conversion element.

In this Example, the arithmetic average roughness Ra was calculated by the image analysis of an image obtained as a height image in an AM-FM mode with an atomic force microscope AFM/SPM (MFP-3D Origin manufactured by Oxford Instruments plc). As measurement conditions, a cantilever OMCL-AC-160TS (manufactured by Olympus Corporation) was used, and measurement was performed in the range of 90 μm×90 μm at a scanning frequency of 1 Hz. In addition, the number of X data was set to 256 and the number of Y data was set to 256.

### [Measurement of Crystal Size of Perovskite Structure in Photoelectric Conversion Layer]

The crystal size of the perovskite structure in the photoelectric conversion layer (hereinafter also referred to as “crystal size”) may be observed by using a SEM. For example, the crystal size may be observed by performing the surface observation of a sample formed up to the photoelectric conversion layer, or performing cross-sectional observation through the cutting of the photoelectric conversion element.

In the present disclosure, the crystal size was measured as described below. First, the upper layer of the photoelectric conversion element was peeled off with a solvent or the like, which did not break the perovskite crystal, to expose the perovskite crystal. Next, the sample with the exposed perovskite crystal was fixed to a stage, and then the crystal size was observed by surface observation with a SEM (apparatus: SmartSEM manufactured by Carl Zeiss Co., Ltd.).

In the observation, the perovskite crystal was distinguished from other layers based on a difference in contrast in an observed image and composition analysis by a SEM-EDX function, and the major axis of the largest perovskite crystal was measured in an image taken at a magnification of 10,000. Such image was taken at three random locations, and the average value of the major axes of the largest perovskite crystals in the respective images was adopted as the crystal size.

### EXAMPLES

The present disclosure is described in more detail below by way of Examples and Comparative Examples. The present disclosure is by no means limited to the following Examples without departing from the gist thereof. In the description of the following Examples, the term “part(s)” is by mass unless otherwise specified.

### Example 1

### <Production of Charge-Transporting Particle 1>

### Step (1)

Under a nitrogen flow atmosphere, 5.46 parts of orthophthalonitrile and 45 parts of α-chloronaphthalene were loaded into a reaction kettle. After that, the mixture was heated so that its temperature was increased to 30° C., followed by the maintenance of the temperature. Next, 3.75 parts of gallium trichloride was loaded into the mixture at the temperature (30° C.). The moisture concentration of the mixed liquid at the time of the loading was 150 ppm.

After that, the temperature of the mixed liquid was increased to 200° C. Next, under a nitrogen flow atmosphere, the mixed liquid was subjected to a reaction at a temperature of 200° C. for 4.5 hours, and was then cooled. The product was filtered when its temperature reached 150° C. The resultant filter residue was subjected to dispersion washing with N,N-dimethylformamide at a temperature of 140° C. for 2 hours, and was then filtered. The resultant filter residue was washed with methanol, and was then dried to provide a chlorogallium phthalocyanine particle in a yield of 71%.

### Step (2)

4.65 Parts of the chlorogallium phthalocyanine particle was dissolved in 139.5 parts of concentrated sulfuric acid at a temperature of 10° C., and the solution was dropped into 620 parts of ice water under stirring so that the particle was reprecipitated, followed by filtration with a filter press under reduced pressure. At this time, No. 5C (manufactured by Advantec Toyo Kaisha, Ltd.) was used as a filter. The resultant wet cake (filter residue) was subjected to dispersion washing with 2% ammonia water for 30 minutes, and was then filtered with the filter press.

Next, the resultant wet cake (filter residue) was subjected to dispersion washing with ion-exchanged water, and then its filtration with the filter press was repeated three times. Finally, the filter residue was freeze-dried to provide a hydroxygallium phthalocyanine particle (hydrous hydroxygallium phthalocyanine particle) having a solid content of 23 mass % in a yield of 71 mass %. The hydroxygallium phthalocyanine particle was dried with a hyper-dry dryer (product name: HD-06R, frequency (oscillatory frequency): 2,455 MHz±15 MHz, manufactured by Biocon (Japan) Ltd.). Thus, a hydroxygallium phthalocyanine (OHGaPc) particle (crystal) having a water content of 1.0 mass % or less was obtained.

### Step (3)

5 Parts of the hydroxygallium phthalocyanine (hereinafter also referred to as “HOGaPc”) particle was mixed with 5 parts of an N-methylformamide solvent, and the mixture was subjected to dispersion treatment for 6 hours with a sand mill (TSG-1/4G-4U, manufactured by Igarashi Machine Production Co., Ltd. (currently AIMEX Co., Ltd.), disc diameter: 70 mm, number of discs: 5) having sealed therein 5 parts of glass beads, followed by filtration and drying to provide a charge-transporting particle 1 (specific gravity: 1.6). The aspect ratio of the particle was 3 or less.

### <Production of Resin Solution 1>

1.0 Gram of polyvinyl butyral (product name: BM-2, manufactured by Sekisui Chemical Co., Ltd., specific gravity: 1.6, glass transition temperature: 95° C.) was dissolved in 19 g of 2-propanol by stirring for 24 hours to provide a resin solution 1.

### [Formation of Electron-Transporting Layer]

Two 100 mm×100 mm square glass substrates with ITO were washed, and a tin(II) oxide colloidal solution (15% water dispersion, manufactured by Alfa Aesar) diluted fivefold was applied thereonto by spin coating, followed by heating at 150° C. for 30 minutes to form an electron-transporting layer as a thin film having a thickness of 16 nm.

### [Formation of Photoelectric Conversion Layer]

8.245 Grams of lead iodide, 2.843 g of methylammonium iodide, and 0.362 g of methylammonium chloride were dissolved in 6.10 g of N,N-dimethylformamide and 7.45 g of N-methyl-2-pyrrolidone, and were stirred for 1 hour to prepare a coating liquid 1 for a photoelectric conversion layer. The coating liquid was applied by die coating with a die head having a width of 30 mm attached on the electron-transporting layer warmed to 55° C., dried by a gas quench method, and finally heated to form a black photoelectric conversion layer composed of MAPbI₃ and having a width of 30 mm, a length of 100 mm, a thickness of 600 nm, and an arithmetic average roughness Ra of 125 nm.

### [Formation of Charge-Transporting Layer]

0.10 Gram of the charge-transporting particle 1 and 0.010 g of a calixarene compound (Japanese Patent Laid-Open No. 2003-207913) were mixed with 10.6 g of 2-propanol, and 11 g of beads (zirconia beads, Torayceram (trademark) zirconia beads, 0.3 mm) were sealed in the mixture, followed by dispersion with a paint shaker (manufactured by Toyo Seiki Co., Ltd.) for 7 hours.

After that, 0.20 g of the resin solution 1 was added thereto, and the mixture was dispersed with the paint shaker again for 6 hours to prepare a coating liquid for a charge-transporting layer. The charge-transporting layer solution was applied by die coating with a die head having a width of 30 mm attached on one photoelectric conversion layer of the two substrates each having formed thereon the photoelectric conversion layer, followed by drying to form a charge-transporting layer having a thickness of 150 nm.

### [Introduction of Second Charge-Transporting Layer]

0.15 Gram of Spiro-OMeTAD serving as a material for a second charge-transporting layer was dissolved in 2.2 g of chlorobenzene. 36 Microliters of an acetonitrile solution obtained by dissolving 0.2 g of lithium bis(trifluoromethanesulfonyl)imide in 0.3 g of acetonitrile and 60 L of 4-tert-butylpyridine (TBP) were added to the chlorobenzene solution, and the contents were mixed.

Further, 58 μL of an acetonitrile solution obtained by dissolving 0.11 g of [tris(2-(1H-pyrazol-1-yl)-4-tert-butylpyridine)cobalt(III) tris(bis(trifluoromethylsulfonyl)imide)] in 0.3 g of acetonitrile was mixed thereinto to prepare a coating liquid 1 for a second charge-transporting layer. The coating liquid was applied onto the above-mentioned charge-transporting layer and onto the photoelectric conversion layer, to which the charge-transporting layer was not applied, by a spin coating method to form a second charge-transporting layer having a thickness of 100 nm.

### [Formation of First Electrode]

In the glass substrate with ITO on which the second charge-transporting layer was formed, three 25 mm square substrates were cut out from the location where the second charge-transporting layer was formed. For each of the resultant 25 mm square substrates, gold electrodes each having a thickness of 80 nm and an area of 0.09 cm² were formed at 10 locations by a vacuum vapor deposition method to provide photoelectric conversion elements. The value of the ratio of the volume of the charge-transporting particle to the volume of an insulating resin in the charge-transporting layer of each of the resultant photoelectric conversion elements was 7.1.

### Example 2

A photoelectric conversion element was obtained in the same manner as in Example 1 except that the glass substrates with ITO used in the formation of the electron-transporting layer were each changed to a square 25 mm on a side. In the formation of the first electrode, a gold electrode was formed on one 25 mm square substrate.

### Example 3

A photoelectric conversion element is obtained in the same manner as in Example 1 except that, in the formation of the photoelectric conversion layer, the wind speed of the gas quenching is increased. In the formation of the photoelectric conversion layer, blackening during the gas quenching is faster than that in Example 1.

### Example 4

A photoelectric conversion element is obtained in the same manner as in Example 3 except that, in the formation of the photoelectric conversion layer, the wind speed of the gas quenching in the step (A-2) is further increased.

### Example 5

A photoelectric conversion element is obtained in the same manner as in Example 1 except that, in the preparation of the coating liquid 1 for a photoelectric conversion layer, methylammonium chloride is not used.

### Example 6

A photoelectric conversion element is obtained in the same manner as in Example 2 except that, in the formation of the photoelectric conversion layer, a photoelectric conversion layer composed of MAPbI₃ is formed by performing the laser co-deposition of lead iodide and methylammonium iodide.

### Example 7

A photoelectric conversion element 7 is obtained in the same manner as in Example 1 except that, in the formation of the photoelectric conversion layer, the die coating is changed to bar coating, and a poor solvent method is used instead of the gas quenching.

### Example 8

A photoelectric conversion element is obtained in the same manner as in Example 1 except that the coating method is changed to bar coating in the formation of the photoelectric conversion layer.

### Example 9

A photoelectric conversion element is obtained in the same manner as in Example 1 except that the coating method is changed to blade coating in the formation of the photoelectric conversion layer.

### Example 10

A photoelectric conversion element is obtained in the same manner as in Example 1 except that the coating method is changed to spray coating in the formation of the photoelectric conversion layer.

### Example 11

A photoelectric conversion element is obtained in the same manner as in Example 1 except that the coating method is changed to ink jet coating in the formation of the photoelectric conversion layer.

### Example 12

A photoelectric conversion element is obtained in the same manner as in Example 1 except that: in the formation of the electron-transporting layer, the glass substrates with ITO to be used are each changed to a film with ITO; and in the formation of the photoelectric conversion layer, the coating method is changed to gravure coating.

### Example 13

A photoelectric conversion element is obtained in the same manner as in Example 1 except that the coating method is changed to screen coating in the formation of the photoelectric conversion layer.

### Example 14

A photoelectric conversion element is obtained in the same manner as in Example 1 except that a film is formed by spin coating instead of the die coating in the formation of the charge-transporting layer.

### Example 15

A photoelectric conversion element is obtained in the same manner as in Example 1 except that a film is formed by blade coating instead of the die coating in the formation of the charge-transporting layer.

### Example 16

A photoelectric conversion element is obtained in the same manner as in Example 1 except that a film is formed by bar coating instead of the die coating in the formation of the charge-transporting layer.

### Example 17

A photoelectric conversion element is obtained in the same manner as in Example 1 except that a film is formed by spray coating instead of the die coating in the formation of the charge-transporting layer.

### Example 18

A photoelectric conversion element is obtained in the same manner as in Example 1 except that a film is formed by ink jet coating instead of the die coating in the formation of the charge-transporting layer.

### Example 19

A photoelectric conversion element is obtained in the same manner as in Example 1 except that: in the formation of the electron-transporting layer, the glass substrates with ITO to be used are each changed to a film with ITO; and in the formation of the charge-transporting layer, a film is formed by gravure coating instead of the die coating.

### Example 20

A photoelectric conversion element is obtained in the same manner as in Example 1 except that a film is formed by screen coating instead of the die coating in the formation of the charge-transporting layer.

### Example 21

A photoelectric conversion element is obtained in the same manner as in Example 9 except that a film is formed by bar coating instead of the die coating in the formation of the charge-transporting layer.

### Example 22

A photoelectric conversion element is obtained in the same manner as in Example 1 except that the drying is performed by a poor solvent method instead of the gas quench method in the formation of the photoelectric conversion layer.

### Example 23

A photoelectric conversion element is obtained in the same manner as in Example 1 except that the drying is performed by air drying without using the gas quench method in the formation of the photoelectric conversion layer.

### Example 24

A photoelectric conversion element is obtained in the same manner as in Example 8 except that the coating method is changed to spin coating in the formation of the charge-transporting layer.

### Example 25

A photoelectric conversion element is obtained in the same manner as in Example 8 except that the coating method is changed to bar coating in the formation of the charge-transporting layer.

### Example 26

A photoelectric conversion element is obtained in the same manner as in Example 8 except that the coating method is changed to blade coating in the formation of the charge-transporting layer.

### Example 27

A photoelectric conversion element is obtained in the same manner as in Example 1 except that a coating liquid obtained by removing a coarse particle from the coating liquid for a charge-transporting layer through centrifugation is used in the formation of the charge-transporting layer. The color of the film to be obtained is lighter than those of other samples.

### Example 28

A photoelectric conversion element is obtained in the same manner as in Example 1 except that the charge-transporting particle 1 is changed to a quinacridone particle.

### Example 29

A photoelectric conversion element is obtained in the same manner as in Example 1 except that the charge-transporting particle 1 is changed to tetraphenylporphyrin (TPP).

### Example 30

A photoelectric conversion element is obtained in the same manner as in Example 1 except that the charge-transporting particle 1 is changed to a particle containing a compound (CuPc) represented by the following formula (Pc-3).

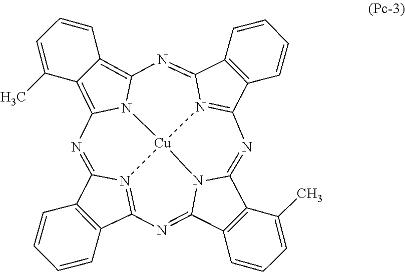

### Example 31

A photoelectric conversion element is obtained in the same manner as in Example 1 except that the polyvinyl butyral is changed to polymethyl methacrylate (PMMA, manufactured by Sigma-Aldrich Co. LLC, glass transition temperature: 100° C.).

### Example 32

A photoelectric conversion element is obtained in the same manner as in Example 1 except that the polyvinyl butyral is changed to polymethyl methacrylate (PMMA, manufactured by Sigma-Aldrich Co. LLC, glass transition temperature: 70° C.).

### Example 33

A photoelectric conversion element is obtained in the same manner as in Example 1 except that the second charge-transporting layer is not formed.

### Example 34

A photoelectric conversion element is produced in the same manner as in Example 1 except that, in the formation of the photoelectric conversion layer, a coating liquid 2 for a photoelectric conversion layer prepared by the following method is used instead of the coating liquid 1 for a photoelectric conversion layer. 0.487 Gram of lead bromide, 1.034 g of formamidinium iodide, 2.903 g of lead iodide, 0.139 g of methylammonium bromide, and 0.364 g of methylammonium chloride are dissolved in 4.25 g of N,N-dimethylformamide and 1.216 g of dimethyl sulfoxide, and are stirred for 1 hour (solution 1). In addition, 0.100 g of cesium iodide is dissolved in 0.285 g of dimethyl sulfoxide, and the solution is stirred for 1 hour (solution 2). After that, the dissolved cesium iodide solution (solution 2) is added to the solution 1 to prepare the coating liquid 2 for a photoelectric conversion layer.

### Example 35

A photoelectric conversion element is produced in the same manner as in Example 1 except that, in the formation of the second charge-transporting layer, a coating liquid 2 for a second charge-transporting layer prepared by dissolving 60 mg of P3HT in 3 mL of chlorobenzene is used instead of the coating liquid 1 for a second charge-transporting layer. The thickness of the second charge-transporting layer is 50 nm.

### Example 36

A photoelectric conversion element is obtained in the same manner as in Example 1 except that the amount of the resin solution 1 to be added is changed to 0.28 g in the formation of the charge-transporting layer. The value of the ratio of the volume of the charge-transporting particle to the volume of the insulating resin in the charge-transporting layer of the photoelectric conversion element to be obtained is 5.0.

### Example 37

A photoelectric conversion element is obtained in the same manner as in Example 1 except that the amount of the resin solution 1 to be added is changed to 0.05 g in the formation of the charge-transporting layer. The value of the ratio of the volume of the charge-transporting particle to the volume of the insulating resin in the charge-transporting layer of the photoelectric conversion element to be obtained is 29.5.

### Example 38

A photoelectric conversion element is obtained in the same manner as in Example 1 except that the amount of the resin solution 1 to be added is changed to 0.40 g in the formation of the charge-transporting layer. The value of the ratio of the volume of the charge-transporting particle to the volume of the insulating resin in the charge-transporting layer of the photoelectric conversion element to be obtained is 3.5. The maximum value of the photoelectric conversion efficiency of each of the photoelectric conversion elements each including the charge-transporting layer to be produced in Examples 1 to 37 is better than the maximum value of the photoelectric conversion efficiency of the photoelectric conversion element including the charge-transporting layer to be produced in Example 38.

### Comparative Example 1

A photoelectric conversion element was obtained in the same manner as in Example 2 except that a film was formed by spin coating instead of the die coating in the formation of the photoelectric conversion layer.

### Comparative Example 2

A photoelectric conversion element is obtained in the same manner as in Example 1 except that the charge-transporting particle 1 is not used in the formation of the charge-transporting layer.

### Comparative Example 3

A photoelectric conversion element is obtained in the same manner as in Example 1 except that the insulating resin is not used in the formation of the charge-transporting layer.

### Comparative Example 4

A photoelectric conversion element is obtained in the same manner as in Example 1 except that, P3HT is used as a conductive resin instead of the insulating resin in the formation of the charge-transporting layer.

### Comparative Example 5

A photoelectric conversion element is obtained in the same manner as in Comparative Example 1 except that the drying is performed by a poor solvent method instead of the gas quenching in the formation of the photoelectric conversion layer.

### Comparative Example 6

A photoelectric conversion element was obtained in the same manner as in Example 1 except that, in the formation of the charge-transporting layer, a solution obtained by dissolving 0.25 part of phenethylamine hydroiodide in 78.5 parts of 2-propanol was applied instead of the coating liquid for a charge-transporting layer and dried by air drying.

Part of the production conditions of the photoelectric conversion elements produced in Examples 1 to 38 and Comparative Examples 1 to 6 described above are shown in Table 2.

### [Evaluation]

### [Analysis of Compound Amount]

The electrode surface of each of the photoelectric conversion elements was peeled off so that the surface of the charge-transporting layer thereof was exposed. The surface of the charge-transporting layer was wiped with a cotton swab or the like with a solvent, dissolved in deuterated sulfuric acid, and subjected to ¹H-NMR measurement (apparatus: AVANCE III 500 manufactured by BRUKER). In addition, the peeled-off charge-transporting layer components were subjected to mass and structure analyses through GPC, MALDI-TOF-MS, IR, gas chromatography, or elemental analysis, such as XPS or EDX, to determine the presence of a compound.

### [Observation of Void in Photoelectric Conversion Layer]

The presence or absence of a void in the photoelectric conversion layer of each of the photoelectric conversion elements was recognized by the method described above. That is, after the photoelectric conversion element had been cut and fixed to an inclined sample stage, the presence or absence was recognized by cross-sectional observation with a SEM (apparatus: SmartSEM manufactured by Carl Zeiss Co., Ltd.). In the observation, the charge-transporting layer was distinguished from other layers based on a difference in contrast in an observed image and composition analysis by a SEM-EDX function, and the number of voids was observed in an image taken at a magnification of 10,000. Such image was taken at ten random locations, and the total number of observed voids was counted. The results are shown in Table 3.

### [Observation of Film Formation Area of Photoelectric Conversion Layer]

The film formation area of the photoelectric conversion layer was recognized by the method described above. That is, first, a portion where the photoelectric conversion layer was formed was visually observed. Next, the length of each side of the resultant photoelectric conversion layer was measured with a ruler, and the film formation area was calculated.

### [Measurement of Arithmetic Average Roughness Ra of Photoelectric Conversion Layer]

The measurement of the arithmetic average roughness Ra of the photoelectric conversion layer of each of the photoelectric conversion elements was performed by the following method. For a sample formed up to the photoelectric conversion layer, an arithmetic average roughness Ra was calculated by the image analysis of an image obtained as a height image in an AM-FM mode with an atomic force microscope AFM/SPM (MFP-3D Origin manufactured by Oxford Instruments). Measurement conditions were as follows: the measurement was performed with a cantilever OMCL-AC-160TS (manufactured by Olympus Corporation) over a range of 90 μm×90 μm at a scanning frequency of 1 Hz.

In addition, the number of X data was set to 256, and the number of Y data was set to 256. In Example 1, it was recognized that the Ra of the sample formed up to the photoelectric conversion layer was the same as the value measured by the method described above after the removal of the first electrode and the charge-transporting layer from the photoelectric conversion element. The results are shown in Table 3.

### [Measurement of Crystal Size of Perovskite Structure in Photoelectric Conversion Layer]

The crystal size of a perovskite structure in each photoelectric conversion layer was measured by the method described above. That is, first, the upper layer of each of the photoelectric conversion elements was peeled off with a solvent or the like, which did not break the perovskite crystal, to expose the perovskite crystal. Next, the sample with the exposed perovskite crystal was fixed to a stage, and then the crystal size was observed by surface observation with a SEM (apparatus: SmartSEM manufactured by Carl Zeiss Co., Ltd.).

In the observation, the perovskite crystal was distinguished from other layers based on a difference in contrast in an observed image and composition analysis by a SEM-EDX function, and the major axis of the largest perovskite crystal was measured in an image taken at a magnification of 10,000. Such image was taken at three random locations, and the average value of the major axes of the largest perovskite crystals in the respective images was adopted as the crystal size. The results are shown in Table 3.

### [Measurement of Average Thickness]

The average thicknesses of the photoelectric conversion layer and charge-transporting layer of each of the photoelectric conversion elements were recognized by the method described above. That is, after the photoelectric conversion element had been cut and fixed to an inclined sample stage, the average thicknesses were recognized by cross-sectional observation with a SEM (apparatus: SmartSEM manufactured by Carl Zeiss Co., Ltd.).

In the observation, the charge-transporting layer was distinguished from other layers based on a difference in contrast in an observed image and composition analysis by a SEM-EDX function, and the average value of the thicknesses of the charge-transporting layer portion was measured by the image processing of an image taken at a magnification of 50,000. Such image was taken at five random locations, and the average of the five average values was adopted as the average thickness of the charge-transporting layer. The results are shown in Table 3.

### [Evaluation of Mass Productivity]

With regard to the presence or absence of mass productivity, as described in the “DESCRIPTION OF THE EMBODIMENTS” section, it was judged that mass productivity was not provided when a photoelectric conversion layer was formed by spin coating. The results are shown in Table 2.

### [Evaluation of Uniformity]

A power source (236 model manufactured by Keithley Instruments) was connected between the electrodes of each of the photoelectric conversion element on which the charge-transporting layer was formed and the photoelectric conversion element on which the charge-transporting layer was not formed produced in each of Examples and Comparative Examples. A solar simulator (manufactured by Yamashita Denso Corporation) having an intensity of 114 mW/cm² was used to apply a certain amount of light, and photoelectric conversion efficiency was measured by measuring a generated current and voltage. The measurement was performed for each of 10 electrodes for all produced 25 mm square samples of each photoelectric conversion element.

For each of the sample on which the charge-transporting layer was formed and the photoelectric conversion element on which the charge-transporting layer was not formed, a difference between the highest photoelectric conversion efficiency and the lowest photoelectric conversion efficiency was calculated and defined as ΔPCE₁ and ΔPCE₀, respectively. When the photoelectric conversion layer had a large film formation area and a plurality of 25 mm square photoelectric conversion elements were cut out in the formation of the first electrode, ΔPCE₁ and ΔPCE₀ were calculated by using the highest photoelectric conversion efficiency and lowest photoelectric conversion efficiency of the plurality of photoelectric conversion elements. For example, the photoelectric conversion element produced in Example 1 had three samples on each of which the charge-transporting layer was formed, and hence a difference between the highest value and lowest value of photoelectric conversion efficiencies measured at a total of 30 electrodes was defined as ΔPCE₁. Similarly, the element also had three samples on each of which the charge-transporting layer was not formed, and hence a difference between the highest value and lowest value of photoelectric conversion efficiencies measured at a total of 30 electrodes was defined as ΔPCE₀. A difference between the resultant ΔPCE₁ and ΔPCE₀ was defined as ΔΔPCE=ΔPCE₀−ΔPCE₁, and it was recognized by the value of ΔΔPCE that uniformity was improved by the presence of the charge-transporting layer. The results are shown in Table 3.

When ΔΔPCE was 2% or more, it was judged that the effect of the present disclosure was exhibited (the uniformity of the photoelectric conversion efficiency was secured). However, for Comparative Example 2, the photoelectric conversion efficiency of the sample on which the charge-transporting layer was formed was very low, and the performance thereof as a photoelectric conversion element was not exhibited. Accordingly, it was judged that a photoelectric conversion element was not obtained, and the calculation of ΔΔPCE was not performed.

TABLE 2

| | Photoelectric conversion layer | | | Charge-transporting layer | | | | |
|---|---|---|---|---|---|---|---|---|
| | | | Film | Film | | | | |
| | | | physical | formation | Charge- | | Glass | Second |
| | Film formation method | | properties | method | transporting | Insulating | transition | charge- |
| | Coating | Drying | Area | Coating | particle | resin | temperature | transporting |
| Example | method | method | [cm²] | method | Material | Material | [° C.] | layer |
| Example 1 | Die | Gas | 30 | Die | HOGaPc | BM-2 | 95 | Present |
| Example 2 | Die | Gas | 6 | Die | HOGaPc | BM-2 | 95 | Present |
| Example 3 | Die | Gas | 30 | Die | HOGaPc | BM-2 | 95 | Present |
| Example 4 | Die | Gas | 30 | Die | HOGaPc | BM-2 | 95 | Present |
| Example 5 | Die | Gas | 30 | Die | HOGaPc | BM-2 | 95 | Present |
| Example 6 | (Vapor deposition) | | 6 | Die | HOGaPc | BM-2 | 95 | Present |
| Example 7 | Bar | Poor | 30 | Die | HOGaPc | BM-2 | 95 | Present |
| | | solvent | | | | | | |
| Example 8 | Bar | Gas | 30 | Die | HOGaPc | BM-2 | 95 | Present |
| Example 9 | Blade | Gas | 30 | Die | HOGaPc | BM-2 | 95 | Present |
| Example 10 | Spray | Gas | 30 | Die | HOGaPc | BM-2 | 95 | Present |
| Example 11 | Ink jet | Gas | 30 | Die | HOGaPc | BM-2 | 95 | Present |
| Example 12 | Gravure | Gas | 30 | Die | HOGaPc | BM-2 | 95 | Present |
| Example 13 | Screen | Gas | 30 | Die | HOGaPc | BM-2 | 95 | Present |
| Example 14 | Die | Gas | 30 | Spin | HOGaPc | BM-2 | 95 | Present |
| Example 15 | Die | Gas | 30 | Blade | HOGaPc | BM-2 | 95 | Present |
| Example 16 | Die | Gas | 30 | Bar | HOGaPc | BM-2 | 95 | Present |
| Example 17 | Die | Gas | 30 | Spray | HOGaPc | BM-2 | 95 | Present |
| Example 18 | Die | Gas | 30 | Ink jet | HOGaPc | BM-2 | 95 | Present |
| Example 19 | Die | Gas | 30 | Gravure | HOGaPc | BM-2 | 95 | Present |
| Example 20 | Die | Gas | 30 | Screen | HOGaPc | BM-2 | 95 | Present |
| Example 21 | Blade | Gas | 30 | Bar | HOGaPc | BM-2 | 95 | Present |
| Example 22 | Die | Poor | 30 | Die | HOGaPc | BM-2 | 95 | Present |
| | | solvent | | | | | | |
| Example 23 | Die | Air drying | 30 | Die | HOGaPc | BM-2 | 95 | Present |
| Example 24 | Bar | Gas | 30 | Spin | HOGaPc | BM-2 | 95 | Present |
| Example 25 | Bar | Gas | 30 | Bar | HOGaPc | BM-2 | 95 | Present |
| Example 26 | Bar | Gas | 30 | Blade | HOGaPc | BM-2 | 95 | Present |
| Example 27 | Die | Gas | 30 | Die | HOGaPc | BM-2 | 95 | Present |
| Example 28 | Die | Gas | 30 | Die | Quinacridone | BM-2 | 95 | Present |
| Example 29 | Die | Gas | 30 | Die | Tetraphenylporphyrin | BM-2 | 95 | Present |
| Example 30 | Die | Gas | 30 | Die | CuPc | BM-2 | 95 | Present |
| Example 31 | Die | Gas | 30 | Die | HOGaPc | PMMA | 100 | Present |
| Example 32 | Die | Gas | 30 | Die | HOGaPc | PMMA | 70 | Present |
| Example 33 | Die | Gas | 30 | Die | HOGaPc | BM-2 | 95 | Absent |
| Example 34 | Die | Gas | 30 | Die | HOGaPc | BM-2 | 95 | Present |
| Example 35 | Die | Gas | 30 | Die | HOGaPc | BM-2 | 95 | Present |
| Example 36 | Die | Gas | 30 | Die | HOGaPc | BM-2 | 95 | Present |
| Example 37 | Die | Gas | 30 | Die | HOGaPc | BM-2 | 95 | Present |
| Example 38 | Die | Gas | 30 | Die | HOGaPc | BM-2 | 95 | Present |
| Comparative | Spin | Gas | 6 | Die | HOGaPc | BM-2 | 95 | Present |
| Example 1 | | | | | | | | |
| Comparative | Die | Gas | 30 | Die | Absent | BM-2 | 95 | Present |
| Example 2 | | | | | | | | |
| Comparative | Die | Gas | 30 | Die | HOGaPc | Absent | 95 | Present |
| Example 3 | | | | | | | | |
| Comparative | Die | Gas | 30 | Die | HOGaPc | (P3HT) | 95 | Present |
| Example 4 | | | | | | | | |
| Comparative | Spin | Poor | 6 | Die | HOGaPc | BM-2 | 95 | Present |
| Example 5 | | solvent | | | | | | |
| Comparative | Die | Gas | 30 | Die | Absent | PEAI | 95 | Present |
| Example 6 | | | | | | | | |

TABLE 3

| | Film state | | | | | | |
|---|---|---|---|---|---|---|---|
| | | | | Charge- | | | |
| | | | | transporting | | | |
| | Photoelectric conversion layer | | | layer | Characteristics | | |
| | Void | Ra | Crystal size | Thickness | Mass | ΔPCE₁ | ΔΔPCE |
| Example | [number] | [nm] | [μm] | [nm] | productivity | [%] | [%] |
| Example 1 | 11 | 125 | 3.5 | 150 | Present | 1.0 | 5.8 |
| Example 2 | 8 | 72 | 3.1 | 150 | Present | 0.4 | 2.2 |
| Example 3 | 0 | 52 | 2.7 | 150 | Present | 0.8 | 4.5 |
| Example 4 | 0 | 26 | 1.3 | 150 | Present | 0.5 | 3.0 |
| Example 5 | 2 | 46 | 0.7 | 150 | Present | 0.8 | 5.1 |
| Example 6 | 0 | 65 | 1.1 | 150 | Present | 0.7 | 4.5 |
| Example 7 | 2 | 41 | 1.3 | 150 | Present | 1.2 | 7.2 |
| Example 8 | 8 | 113 | 2.5 | 150 | Present | 1.1 | 6.8 |
| Example 9 | 7 | 93 | 2.4 | 150 | Present | 1.1 | 6.3 |
| Example 10 | 7 | 102 | 2.5 | 150 | Present | 1.3 | 7.7 |
| Example 11 | 7 | 116 | 3.2 | 150 | Present | 1.3 | 7.9 |
| Example 12 | 7 | 70 | 2.3 | 150 | Present | 1.0 | 6.2 |
| Example 13 | 7 | 105 | 2.4 | 150 | Present | 1.1 | 6.4 |
| Example 14 | 11 | 125 | 3.5 | 150 | Present | 2.7 | 4.1 |
| Example 15 | 11 | 125 | 3.5 | 150 | Present | 1.4 | 5.4 |
| Example 16 | 11 | 125 | 3.5 | 150 | Present | 2.3 | 4.5 |
| Example 17 | 11 | 125 | 3.5 | 150 | Present | 1.8 | 5.0 |
| Example 18 | 11 | 125 | 3.5 | 150 | Present | 1.9 | 4.9 |
| Example 19 | 11 | 125 | 3.5 | 150 | Present | 1.3 | 5.5 |
| Example 20 | 11 | 125 | 3.5 | 150 | Present | 1.3 | 5.5 |
| Example 21 | 7 | 93 | 2.4 | 150 | Present | 2.5 | 4.9 |
| Example 22 | 1 | 45 | 1.2 | 150 | Present | 1.0 | 6.1 |
| Example 23 | 9 | 143 | 3.8 | 150 | Present | 1.2 | 7.5 |
| Example 24 | 8 | 113 | 2.5 | 150 | Present | 2.9 | 5.0 |
| Example 25 | 8 | 113 | 2.5 | 150 | Present | 2.6 | 5.3 |
| Example 26 | 8 | 113 | 2.5 | 150 | Present | 3.2 | 4.7 |
| Example 27 | 11 | 125 | 3.5 | 50 | Present | 2.5 | 4.3 |
| Example 28 | 11 | 125 | 3.5 | 150 | Present | 1.8 | 5.0 |
| Example 29 | 11 | 125 | 3.5 | 150 | Present | 1.6 | 5.2 |
| Example 30 | 11 | 125 | 3.5 | 150 | Present | 1.5 | 5.3 |
| Example 31 | 11 | 125 | 3.5 | 150 | Present | 1.5 | 5.3 |
| Example 32 | 11 | 125 | 3.5 | 150 | Present | 1.4 | 5.4 |
| Example 33 | 11 | 125 | 3.5 | 150 | Present | 2.9 | 3.9 |
| Example 34 | 11 | 125 | 3.5 | 150 | Present | 0.8 | 4.5 |
| Example 35 | 11 | 125 | 3.5 | 150 | Present | 1.1 | 6.6 |
| Example 36 | 11 | 125 | 3.5 | 150 | Present | 0.9 | 5.9 |
| Example 37 | 11 | 125 | 3.5 | 150 | Present | 4.3 | 2.5 |
| Example 38 | 11 | 125 | 3.5 | 150 | Present | 0.7 | 6.1 |
| Comparative | 0 | 30 | 0.8 | 150 | Absent | 0.3 | 0.2 |
| Example 1 | | | | | | | |
| Comparative | 11 | 125 | 3.5 | 150 | Present | — | — |
| Example 2 | | | | | | | |
| Comparative | 11 | 125 | 3.5 | 150 | Present | 5.2 | 1.6 |
| Example 3 | | | | | | | |
| Comparative | 11 | 125 | 3.5 | 150 | Present | 6.1 | 0.7 |
| Example 4 | | | | | | | |
| Comparative | 0 | 26 | 0.5 | 150 | Absent | 0.2 | 0.0 |
| Example 5 | | | | | | | |
| Comparative | 11 | 125 | 3.5 | 150 | Present | 6.2 | 0.6 |
| 6Example | | | | | | | |

According to the present disclosure, there can be provided a method of producing a photoelectric conversion element that achieves both mass productivity and the uniformity of photoelectric conversion efficiency.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A method of producing a photoelectric conversion element, the method comprising:
a step (A) of forming a photoelectric conversion layer containing a crystal having a perovskite structure by using a large-area film formation method; and
a step (B) of forming a charge-transporting layer by applying a paint for a charge-transporting layer onto a surface of the photoelectric conversion layer and then drying the paint,
wherein the surface of the photoelectric conversion layer has an arithmetic average roughness of 40 nm or more, and
wherein the paint for a charge-transporting layer contains a charge-transporting particle and an insulating resin.

2. The method of producing a photoelectric conversion element according to claim 1, wherein, in the step (A), the photoelectric conversion layer has a film formation area of 20 cm² or more.

3. The method of producing a photoelectric conversion element according to claim 1, wherein the photoelectric conversion layer has a void.

4. The method of producing a photoelectric conversion element according to claim 1, wherein the crystal has a size of 1.0 μm or more.

5. The method of producing a photoelectric conversion element according to claim 1, wherein the step (A) comprises:
a step (A-1) of uniformly applying a paint for a photoelectric conversion layer; and
a step (A-2) of drying the paint for a photoelectric conversion layer.

6. The method of producing a photoelectric conversion element according to claim 5, wherein the step (A-1) comprises a coating process by any one coating method selected from the group consisting of die coating, blade coating, roll coating, spray coating, ink jet coating, gravure coating and screen coating.

7. The method of producing a photoelectric conversion element according to claim 5, wherein the step (A-2) is free of a process of applying a poor solvent onto a raw material for the crystal.

8. The method of producing a photoelectric conversion element according to claim 5, wherein the step (A-2) comprises a process of drying the paint for a photoelectric conversion layer by blowing a gas through use of a unit for blowing a gas.

9. A method of producing a photoelectric conversion element, the method comprising:
a step (A) of forming a photoelectric conversion layer containing a crystal having a perovskite structure by using a large-area film formation method; and
a step (B) of forming a charge-transporting layer by applying a paint for a charge-transporting layer onto a surface of the photoelectric conversion layer and then drying the paint,
wherein the step (A) comprises:
a step (A-1) of uniformly applying a paint for a photoelectric conversion layer; and
a step (A-2) of drying the paint for a photoelectric conversion layer, and
wherein the step (A-2) comprises a process of drying the paint for a photoelectric conversion layer by blowing a gas through use of a unit for blowing a gas.

10. The method of producing a photoelectric conversion element according to claim 9, wherein the step (A-1) comprises a coating process by any one coating method selected from a group consisting of die coating, blade coating, roll coating, spray coating, ink jet coating, gravure coating and screen coating.

11. The method of producing a photoelectric conversion element according to claim 9, wherein the step (A-2) is free of a process of applying a poor solvent onto a raw material for the crystal.

12. The method of producing a photoelectric conversion element according to claim 9, wherein the step (B) comprises the same coating method as that in the step (A-1).

13. The method of producing a photoelectric conversion element according to claim 1, wherein the step (B) comprises a coating process by any one coating method selected from the group consisting of die coating, blade coating, roll coating, spray coating, ink jet coating, gravure coating and screen coating.

14. The method of producing a photoelectric conversion element according to claim 1, wherein the charge-transporting particle contains a cyclic conjugated compound in which a plurality of pyrrole rings are bonded by conjugated bonds.

15. The method of producing a photoelectric conversion element according to claim 1, wherein the charge-transporting particle contains a phthalocyanine compound.

16. The method of producing a photoelectric conversion element according to claim 1, wherein the charge-transporting particle contains a hydroxygallium phthalocyanine compound.

17. The method of producing a photoelectric conversion element according to claim 1, wherein the insulating resin has a glass transition temperature of less than 100° C.

18. The method of producing a photoelectric conversion element according to claim 1, wherein the insulating resin is one of a polyvinyl acetal resin or a polyvinyl butyral resin.

19. The method of producing a photoelectric conversion element according to claim 1, wherein the photoelectric conversion element comprises a second charge-transporting layer free of a charge-transporting particle on the charge-transporting layer.

20. The method of producing a photoelectric conversion element according to claim 1, wherein, in the charge-transporting layer, a value of a ratio of a volume of the charge-transporting particle to a volume of the insulating resin is 5 to 30.
